(19) United States
(12) Patent Application Publication
(10) Pub. No.: US 2011/0008356 A1
(43) Pub. Date: Jan. 13, 2011

(54) IL-17 HETEROMERIC RECEPTOR COMPLEX

(75) Inventors: Jacques J. Peschon, Seattle, WA (US); Joel Tocker, Issaquah, WA (US); Dean Toy, Newcastle, WA (US)

Correspondence Address: AMGEN INC.;LAW DEPARTMENT, 1201 AMGEN COURT WEST, SEATTLE, WA, 98119, US

(73) Assignee: AMGEN, INC., Thousand Oaks, CA (US)

(21) Appl. No.: 12/664,234

(22) PCT Filed: Jun. 13, 2008
(86) PCT No.: PCT/US2008/007460

§ 371 Date: Sep. 23, 2010

Related U.S. Application Data
(60) Provisional application No. 60/943,779, filed on Jun. 13, 2007.

(51) Int. Cl. A61K 39/395; C07K 16/18; A61P 37/02

(52) U.S. Cl. 424/142.1; 530/388.15; 424/158.1

(57) ABSTRACT

The present invention relates to Interleukin-17 ligand and receptor family members and the discovery that IL-17 receptor A and IL-17 receptor C form a heteromeric receptor complex that is biologically active.

### FIELD OF THE INVENTION

The present invention relates to Interleukin-17 ligand and receptor family members and the discovery that IL-17 receptor A and IL-17 receptor C form a heteromeric complex that is biologically active. Antagonists of the IL-17RA-IL-17RC heteromeric receptor complex and methods of use are disclosed.

### BACKGROUND OF THE INVENTION

Interleukin-17, which is also known as and referred to herein as IL-17 and/or IL-17A, is an inflammatory cytokine that induces the production of cytokines and other mediators leading to diseases and physiological effects such as inflammation, cartilage degradation, and bone resorption. IL-17RA is a ubiquitously expressed receptor identified as a mammalian counterstructure for HVS13 and subsequently shown to bind IL-17A with high affinity (Yao, et al., 1995, Immunity 3:811-821). Leukocytes from mice lacking IL-17RA fail to bind IL-17A and antibodies against IL-17RA inhibit the activity of IL-17A on human epithelial cells. These data indicate that at least IL-17RA is involved in IL-17A function (Ye, et al., 2001, J. Exp. Med. 194:519-527).

IL-17A is expressed by a unique lineage of CD4 positive T cells (T_{H}-17) that develop in response to IL-23, in particular under conditions in which T_{H}1 and T_{H}2 development are suppressed (Harrington, et al., 2005, Nat. Immunol. 6:1123-1132; Langrish, et al., 2005, J. Exp. Med. 201:233-240; Park, et al., 2005, Nat. Immunol. 6:1133-1141; Aggarwal, et al., 2003, J. Biol. Chem. 278:1910-1914). Several observations suggest that IL-17A is a key mediator of autoimmune disorders and plays a role in host defense. Recent work indicates that IL-17A is a mediator of autoimmune disorders, including rheumatoid arthritis, psoriasis, inflammatory bowel disease, multiple sclerosis, and asthma, and plays a role in host defense (Kolls, et al., 2004, Immunity 21:467-476; Koenders, et al., 2005, A. J. Pathol 167:141-149; Lubberts, et al., 2005, J. Immunol. 175:3360-3368; Koenders, et al., 2005, Arthritis Rheum. 52:3239-3247; Nakae, et al., 2003, J. Immunol. 171:6173-6177).

An examination of mRNA expression of IL-17A in psoriasis patients showed that IL-17A is upregulated in psoriatic lesions compared with skin from normal controls (Li et al., 2004, Huazhong Univ. Sci. Technolog. Med. Sci. 24:294-296). IL-17A expression is detected in active mucosa of patients with Crohns Disease and ulcerative colitis and the number of IL-17A positive cells are increased in the colonic mucosa of those with active disease compared to inactive patients. IL-17A levels were also found to be increased in the serum of IBD patients (Fujino et al., 2003, Gut. 52:65-70). Microarray analysis of multiple sclerosis lesions obtained at autopsy showed increased transcripts of IL-17A compared to controls without nervous system pathology. IL-17A released from activated CD4+ T cells may play a role in asthma through recruitment and activation of airway neutrophils (Linden et al., 2000 Eur Respir J. May 15(5):973-7). In humans, increased number of cells positive for IL-17A were found in the bronchoalveolar lavage fluid of asthmatics compared to non-asthmatic volunteers with an increase in the peripheral eosinophil IL-17A levels (Molet et al., 2001, J. Allergy Clin. Immunol. 108:430-438).

At the time of their discovery, IL-17A and IL-17RA were structurally unique and did not fall into any known cytokine or cytokine receptor families. More recently, five additional IL-17-like ligands (IL-17B-F) and four additional IL-17R-like receptors (IL-17RB-E) have been identified (Kolls, J. K., et al., 2004 Immunity 21:467-476). The most recently identified ligand, IL-17F, shares the most sequence identity to IL-17A (˜50%) and is dependent on IL-17RA for function (McAllister et al., 2005 J. Immunol. 175:404-412; Kawaguchi, M., et al., 2001 J. Immunol. 167:4430-4435; Hymowitz, S. G., et al., 2001 EMBO J. 20:5532-5341; Starnes, T., et al., 2001 J. Immunol. 167:4137-4140).

The IL-17F response is mediated by the IL-17 receptor, which is also known as and referred to herein as IL-17R, IL-17 receptor A, and/or IL-17RA. IL-17F has ˜50% sequence identity to IL-17A and has similar in vitro activities as IL-17A. Recent studies have suggested that IL-17F plays a role in the induction of neutrophilia in the lungs and in the exacerbation of antigen-induced pulmonary inflammation. (Oda et al., 2006, American J. Resp. Crit. Care Medicine, Jan. 15, 2006). Additional studies indicate that IL-17F plays a role in G-CSF production by lung microvascular endothelial cells stimulated with IL-1β and/or TNF-α (Numasaki et al., 2004, Immunol Lett 95:97-104).

The biologic activities of IL-17A and IL-17F are dependent upon IL-17 receptor family members. More recently, a novel member of the IL-17 receptor family, IL-17 receptor C (IL-17RC), was shown to bind IL-17A and IL-17F with high affinity (approximately 50 pM and 100 pM, respectively) (Levin 18^{th} IMGC 2004). However, the observations that IL-17RA deficiency and IL-17RA antibody neutralization ablate both IL-17A and IL-17F function suggest that IL-17RC cannot deliver an IL-17A or IL-17F signal in the absence of IL-17RA (McAllister et al., 2005 J. Immunol. 175:404-412). Additionally, forced expression of IL-17RC in IL-17RA deficient cells does not restore IL-17A or IL-17F function (Toy et al., 2006, J. Immunol. 177:36-39).

Based on crystallographic analyses, the homodimeric cytokines IL-17A and IL-17F have structural features in common with cystiene knot family growth factors (Hymowitz, et al., 2001, EMBO J. 20:5532-5341). Members of this ligand family are also homodimeric, and have been shown to bind and signal through both homodimeric and heteromeric counterstructures (Lu, et al., 2005, Nat. Rev. Neurosci. 6: 603-614; Barker, 2004, Neuron 42:529-533). Recent evidence suggests that IL-17RA can multimerize, independent of ligand, although the role of IL-17RA oligomerization in delivering an IL-17A signal is unknown. Furthermore, additional subunits for the IL-17RA, or any of the IL-17R family members, have not been described. Here, we show for the first time that IL-17RA forms a heteromeric receptor complex with IL-17RC and that the biological activity of IL-17 ligand family members is dependent on this heteromeric receptor complex. This, and many other aspects of the various embodiments of the invention, are provided.

### BRIEF DESCRIPTION OF THE FIGURES

FIG. 1A is a bar graph depicting data from ELISA assays on supernatants from immortalized wild-type fibroblasts (+/+), IL-17RA-deficient fibroblasts (−/−), and IL-17RA-deficient fibroblasts stably transduced with retroviruses encoding mouse IL-17RA (−/−muRA) or human IL-17A (huIL-17) or IL-17F (huIL-17F) for 18 hrs.; culture supernatants were assayed for the presence of CXCL1 by ELISA. Cultures were analyzed in triplicate. Averages and SEMs are depicted.

FIG. 1B shows flow cytometry data of immortalized wild-type fibroblasts (+/+), IL-17RA-deficient fibroblasts (−/−), and IL-17RA-deficient fibroblasts stably transduced with retroviruses encoding mouse IL-17RA (−/−muRA), human IL-17RA (−/−huRA), human IL-17RC (−/−huRC), both human IL-17RA and human IL-17RC (−/−huRAhuRC), or a cytoplasmic domain deletion variant of human IL-17RC (−/−huRCΔcyt) were analyzed by flow cytometry by staining with 1 ug/ml human IL-17A:Fc (shaded histograms) or negative control Fc (open histograms).

FIG. 2A is a bar graph depicting data from ELISA assays on supernatants from Immortalized wild type (+/+) and IL-17RA-deficient fibroblasts stably transduced with retroviruses encoding human IL-17RC (−/−huRC), human IL-17RA (−/−huRA) or both human IL-17RA and human IL-17RC (−/−huRAhuRC) were stimulated in the presence of 50 ng/ml human IL-17A or IL-17F for 18 hours and culture supernatants assayed for the presence of CXCL1 by ELISA. Cultures were analyzed in triplicate. Averages and SEMs (standard error around the means) are depicted.

FIG. 2B is a bar graph depicting data from ELISA assays on supernatants from wild-type mouse fibroblasts that were stimulated for 18 hrs. with 50 ng/ml mouse IL-17A in the presence of the indicated concentrations of a control IgG, a mAb against IL-17RA (αIL-17R), or a polyclonal Ab against mouse IL-17RC (αIL-17RC); culture supernatants were assayed for the presence of CXCL1 by ELISA. Cultures were analyzed in triplicate. Averages and SEMs are depicted.

FIG. 3A show flow cytometry data from immortalized IL-17RA deficient fibroblasts that were stably transduced with retroviruses encoding cytoplasmic deletion variants of human IL-17RC (huRCΔcyt) or human IL-17RA (huRAΔcyt) and analyzed by flow cytometry by staining with 1 ug/ml human IL-17A:Fc (shaded histograms) or a negative control Fc (open histograms).

FIG. 3B is a bar graph depicting data from ELISA assays on supernatants from immortalized IL-17RA deficient fibroblasts that were stably transduced with retroviruses encoding human IL-17RA (−/−huRA), both human IL-17RA and human IL-17RC (−/− huRAhuRC), or both human IL-17RA plus human IL-17RC cytoplasmic deletion mutant (−/− huRAhuRCΔcyt) were stimulated in the presence of 50 ng/ml human IL-17A or IL-17F for 18 hours and culture supernatants assayed for the presence of CXCL1 by ELISA. Cultures were analyzed in triplicate. Averages and SEMs are depicted.

FIG. 3C is a bar graph depicting data from ELISA assays on supernatants from immortalized IL-17RA deficient fibroblasts were stably transduced with retroviruses encoding human IL-17RC (−/−huRC), both human IL-17RA and human IL-17RC (−/−huRAhuRC), or human IL-17R cytoplasmic deletion mutant plus human IL-17RC (−/−huRAΔcy^{t}huRC) were stimulated in the presence of 50 ng/ml human IL-17A or IL-17F for 18 hours and culture supernatants assayed for the presence of CXCL1 by ELISA. Cultures were analyzed in triplicate. Averages and SEMs are depicted.

FIG. 4A is a Western blot of HEK 293 cells that were transiently transfected with human IL-17RA (RA), human IL-17RC(RC), both human IL-17RA and human IL-7RC (RA/RC) or vector alone (EV); cell lysates were analysed by standard Western blot using polyclonals against either human IL-17RA or human IL-17RC as indicated.

FIG. 4B is a Western blot of human IL-17RC that was immunoprecipitated from the indicated transfected HEK 293 lysates using an anti-FLAG™ monoclonal and the resulting immunoprecipitates were analyzed for the presence of IL-17RA by Western blot.

FIG. 4C is a Western blot of human IL-17RA that was immunoprecipitated from the indicated transfected HEK 293 lysates using an anti-IL-17RA monoclonal and the resulting immunoprecipitates were analyzed for the presence of IL-17RC by Western blot.

### DETAILED DESCRIPTION OF THE INVENTION

The section headings used herein are for organizational purposes only and are not to be construed as limiting the subject matter described.

Standard techniques may be used for recombinant DNA, oligonucleotide synthesis, tissue culture and transformation, protein purification etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manual, 3^{rd} ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytical chemistry, organic chemistry, and medicinal and pharmaceutical chemistry described herein are those well known and commonly used in the art. Standard techniques may be used for chemical synthesis, chemical analyses, pharmaceutical preparation, formulation, and delivery and treatment of patients.

The characterization, cloning, and preparation of IL-17RA are described for example in U.S. Pat. No. 6,072,033, issued Jun. 6, 2000, which is incorporated herein by reference in its entirety. The amino acid sequence of the human IL-17RA is shown in SEQ ID NO:10 of U.S. Pat. No. 6,072,033 (GenBank accession number NM_{—}014339). The human IL-17RA has an N-terminal signal peptide with a predicted cleavage site approximately between amino acid 27 and 28. The signal peptide is followed by a 293 amino acid extracellular domain, a 21 amino acid transmembrane domain, and a 525 amino acid cytoplasmic tail. Soluble forms of human IL-17RA (huIL-17RA) that are useful in the methods of the present invention include the extracellular domain (residues 1-320 or residues 28-320 which excludes the signal peptide) or a fragment of the extracellular domain that retains the capacity to bind IL-17A. Other forms of IL-17RA that are useful in the present invention include muteins and variations that are at least between 70% and 99% amino acid identity to the native IL-17RA that retains the capacity to bind IL-17A, as describe in greater detail in U.S. Pat. No. 6,072,033.

IL-17 Receptor C (IL-17RC) and its many isoforms are known in the art, such as those disclosed and described in WO 2001/04304, WO 2002/38764, WO 2001/90358, WO 2002/102994. Further examples include sequences available on public databases, such as, but not limited to GenBank accession no. NP_{—}703190.1. In addition, as described below, IL-17RC may also include biologically active fragments and/or variants.

In certain embodiments of the invention, it has been discovered that IL-17RA associates with IL-17RC to form a heteromeric receptor complex that is biologically active. Thus, certain aspects of the invention are drawn to agents (e.g., antigen binding proteins, as described below) and methods for blocking the association of IL-17RA and IL-17RC and thereby preventing a functional receptor complex from being formed and capable of being activated. By preventing a functional receptor complex from being formed, or having an antagonist that binds the IL-17RA-IL-17RC heteromeric receptor complex, this would reduce or prevent receptor activation and reduce the downstream proinflammatory effects of IL-17RA/IL-17RC activation. Such methods and antigen binding proteins would be useful in the treatment of various inflammation and autoimmune disorders that are influenced by the IL-17/IL-17R pathway. Embodiments of the invention are useful for in vitro assays to screen for antagonists or agonists of the IL-17RA-IL-17RC heteromeric receptor complex. Embodiments of the invention are useful for in vitro assays to identify cells expressing the IL-17RA-IL-17RC heteromeric receptor complex. These are but a few of the many aspects of the various embodiments of the invention described herein.

1. IL-17RA-IL-17RC Antagonists

It has been discovered that IL-17RA associates with IL-17RC to form a heteromeric receptor complex that is biologically active. An IL-17RA-IL-17RC heteromeric receptor complex is defined as a physical association (such as, but not limited to, protein-protein interactions) of IL-17RA and IL-17RC proteins and displayed as a heteromeric receptor complex on the extracellular membrane of cells. This heteromeric receptor complex, at a minimum, is required for IL-17RA and/or IL-17RC activation. It is understood that the IL-17RA-IL-17RC heteromeric receptor complex may further comprise additional accessory proteins. IL-17RA-IL-17RC heteromeric receptor complex activation is effectuated through binding of IL-17 ligand family members, such as, but not limited to, IL-17A and IL-17F. IL-17RA-IL-17RC heteromeric receptor complex activation includes, but is not limited to, initiation of intracellular signaling cascade(s) and downstream events such as gene transcription and translation.

Embodiments are directed to antigen binding proteins that inhibit the association of IL-17RA and IL-17RC in forming an IL-17RA-IL-17RC heteromeric receptor complex. An antigen binding protein may be an antibody, or fragment thereof, that specifically binds an identified target protein, as variously defined herein. An antigen binding protein may be a peptide or polypeptide that specifically binds the identified target protein. Antigen binding proteins that inhibit the association of IL-17RA and IL-17RC in forming an IL-17RA-IL-17RC heteromeric receptor complex are referred to herein as IL-17RA-IL-17RC antagonists. Embodiments of IL-17RA-IL-17RC antagonists may also bind to any part of the IL-17RA-IL-17RC heteromeric receptor complex and inhibit receptor activation.

“Antigen binding protein” as used herein is a protein that specifically binds an identified target protein. “Specifically binds” means that the antigen binding protein has higher affinity for the identified target protein than for any other protein. Typically, “specifically binds” mean that the equilibrium dissociation constant is <10⁻⁷ to 10⁻¹¹ M, or <10⁻⁸ to <10⁻¹⁰ M, or <10⁻⁹ to <10⁻¹⁰ M.

Activating or activation of a receptor is defined herein as the engagement of one or more intracellular signaling pathway(s) and the transduction of intracellular signaling (i.e., signal transduction) in response to a molecule binding to a membrane-bound receptor, such as but not limited to, a receptor:ligand interaction. Signal transduction, as used herein, is the relaying of a signal by conversion from one physical or chemical form to another; for example, in cell biology, the process by which a cell converts an extracellular signal into a response. Exemplary subgenera of the genus of IL-17RA-IL-17RC antagonists comprise antibodies, as variously defined herein; as well as peptides and polypeptides.

“Inhibition” may be measured as a decrease in the association of IL-17RA and IL-17RC proteins in forming a heteromeric receptor complex by at least 10%, 15%, 20%, 25%, 30%, 35%, 40%, 45%, 50%, 55%, 60%, 65%, 70%, 75%, 80%, 85%, 90%, 95%, or 100%. The inhibition of forming a heteromeric receptor complex may be measured by any means known in the art, such as but not limited to the co-immunoprecipitation methods described herein. Other examples include Forster Resonance Energy Transfer (FRET) analysis. In addition, “inhibition” may be measured as a loss of IL-17A and/or IL-17F activation of an IL-17RA-IL-17RC heteromeric receptor complex as measured by biologically relevant readouts, such as but not limited to upregulated gene transcription and/or gene translation, and/or release of various factors associated with activation of the IL-17RA-IL-17RC heteromeric receptor complex, which includes, but is not limited to: IL-6, IL-8, CXCL1, CXCL2, GM-CSF, G-CSF, M-CSF, IL-1β, TNFα, RANK-L, LIF, PGE2, IL-12, MMP3, MMP9, GROα, NO, as well as any other proinflammatory mediator known in the art to be released from any cells expressing IL-17RA and/or IL-17RC.

Other embodiments of an IL-17RA-IL-17RC antagonist are directed to IL-17RA-IL-17RC antagonists that bind to IL-17RA, and partially inhibit or fully inhibit association of IL-17RA with IL-17RC and thereby prevent IL-17RA-IL-17RC heteromeric receptor complex formation and activation through binding of IL-17 ligand family members, such as but not limited to IL-17A and IL-17F. In one embodiment, the IL-17RA-IL-17RC antagonist need not block the binding of IL-17A and/or IL-17F from binding to the IL-17RA-IL-17RC heteromeric receptor complex. In alternative embodiments, the IL-17RA-IL-17RC antagonist may block the binding of IL-17A and/or IL-17F to the IL-17RA-IL-17RC heteromeric receptor complex.

Embodiments of an IL-17RA-IL-17RC antagonist are directed to IL-17RA-IL-17RC antagonists that bind to IL-17RC and partially inhibit or fully inhibit association of IL-17RC with IL-17RA and thereby prevent IL-17RA-IL-17RC heteromeric receptor complex formation and activation through binding of IL-17 ligand family members, such as but not limited to IL-17A and IL-17F. In one embodiment, the IL-17RA-IL-17RC antagonist need not block the binding of IL-17A and/or IL-17F from binding to the IL-17RA-IL-17RC heteromeric receptor complex. In alternative embodiments, the IL-17RA-IL-17RC antagonist may block the binding of IL-17A and/or IL-17F to the IL-17RA-IL-17RC heteromeric receptor complex.

Further embodiments of an IL-17RA-IL-17RC antagonist are directed to IL-17RA-IL-17RC antagonists that bind to both IL-17RC and IL-17RA, and partially inhibit or fully inhibit association of IL-17RA with IL-17RC and thereby prevent IL-17RA-IL-17RC heteromeric receptor complex formation and activation through binding of IL-17 ligand family members, such as but not limited to IL-17A and IL-17F. In one embodiment, the IL-17RA-IL-17RC antagonist need not block the binding of IL-17A and/or IL-17F from binding to the IL-17RA-IL-17RC heteromeric receptor. In alternative embodiments, the IL-17RA-IL-17RC antagonists may block the binding of IL-17A and/or IL-17F to the IL-17RA-IL-17RC heteromeric receptor.

The various embodiments of IL-17RA-IL-17RC antagonists described above include IL-17RA-IL-17RC antagonists that bind to IL-17RA, or IL-17RC, or both IL-17RA and IL-17RC and sterically inhibit the association of IL-17RA with IL-17RC and thereby prevent IL-17RA-IL-17RC heteromeric receptor complex formation.

Alternatively, the various embodiments of IL-17RA-IL-17RC antagonists described above include IL-17RA-IL-17RC antagonists that bind to IL-17RA, or IL-17RC, or both IL-17RA and IL-17RC and induce a conformational alteration in IL-17RA, or IL-17RC, or both IL-17RA and IL-17RC and thereby inhibit the association of IL-17RA with IL-17RC and consequently prevent IL-17RA-IL-17RC heteromeric receptor complex formation.

### 1.1 IL-17RA-IL-17RC Antagonists: Antibodies

Embodiments of IL-17RA-IL-17RC antagonists comprise antibodies, or fragments thereof, as variously defined herein. Accordingly, the IL-17RA-IL-17RC antagonists include polyclonal antibodies, monoclonal antibodies, bispecific antibodies, diabodies, minibodies, domain antibodies, synthetic antibodies (sometimes referred to herein as “antibody mimetics”), chimeric antibodies, humanized antibodies, fully human antibodies, antibody fusions (sometimes referred to as “antibody conjugates”), as well as fragments thereof.

IL-17RA-IL-17RC antagonist antibodies may also comprise single-domain antibodies that comprise dimers of two heavy chains and include no light chains, such as those found in camels and llamas (see, for example Muldermans, et al., 2001, J. Biotechnol. 74:277-302; Desmyter, et al., 2001, J. Biol. Chem. 276:26285-26290).

IL-17RA-IL-17RC antagonist antibodies may comprise a tetramer, or fragments thereof. Each tetramer is typically composed of two identical pairs of polypeptide chains, each pair having one “light” (typically having a molecular weight of about 25 kDa) and one “heavy” chain (typically having a molecular weight of about 50-70 kDa). The amino-terminal portion of each chain includes a variable region is primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. IL-17RA-IL-17RC antagonist antibodies include all such isotypes. For exemplary purposes, antibody fragments include but are not limited to F(ab), F(ab′), F(ab′)2, Fv, and single chain Fv fragments (scfv), as well as single-chain antibodies. IL-17RA-IL-17RC antagonist antibodies may comprise any of the foregoing examples.

The structure of antibodies is well known in the art and need not be reproduced here, but by way of example, the variable regions of the heavy and light chains typically exhibit the same general structure of relatively conserved framework regions (FR) joined by three hypervariable regions, also called complementarity determining regions or CDRs. The CDRs are the hypervariable regions of an antibody (or antigen binding protein, as outlined herein), that are responsible for antigen recognition and binding. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. In some embodiments, the assignment of amino acids to each domain may be in accordance with the definitions of Kabat Sequences of Proteins of Immunological Interest. See, Chothia, et al., 1987, J. Mol. Biol. 196:901-917; Chothia, et al., 1989, Nature 342:878-883.

A “complementary determining region” or “CDR,” as used herein, refers to a binding protein region that constitutes the major surface contact points for antigen binding. A binding protein of the invention may have six CDRs, for example one heavy chain CDR1 (“CDRH1”), one heavy chain CDR1 (“CDRH1”), one heavy chain CDR2 (“CDRH2”), one heavy chain CDR3 (“CDRH3”), one light chain CDR1 (“CDRL1”), one light chain CDR2 (“CDRL2”), one light chain CDR3 (“CDRL3”). CDRH1 typically comprises about five (5) to about seven (7) amino acids, CDRH2 typically comprises about sixteen (16) to about nineteen (19) amino acids, and CDRH3 typically comprises about three (3) to about twenty five (25) amino acids.

CDRL1 typically comprises about ten (10) to about seventeen (17) amino acids, CDRL2 typically comprises about seven (7) amino acids, and CDRL3 typically comprises about seven (7) to about ten (10) amino acids

At a minimum, an IL-17RA-IL-17RC antagonist antibody comprises all or part of a light or heavy chain variable region, or all or part of both a light and heavy chain variable region that specifically binds to IL-17RA, or IL-17RC, or both IL-17RA and IL-17RC. Examples of fragments (i.e., “part”) of variable regions comprise the CDRs. Stated differently, at a minimum, an IL-17RA-IL-17RC antagonist antibody comprises at least one CDR of a variable region, wherein the CDR specifically binds IL-17RA, or IL-17RC, or both IL-17RA and IL-17RC. In alternative embodiments, an IL-17RA-IL-17RC antagonist antibody comprises at least two, or at least three, or at least four, or at least five, or at least all six CDRs of a/the variable region(s), wherein at least one of the CDRs specifically binds IL-17RA, or IL-17RC, or both IL-17RA and IL-17RC. The CDR may be from a heavy or light chain, and may be one of any of the three CDRs within each chain, that is, the CDRs are each independently selected from CDRH1, CDRH2, CDRH3, CDRL1, CDRL2 and CDRL3.

Embodiments of the IL-17RA-IL-17RC antagonist antibodies may comprise a scaffold structure into which useful CDR(s) are grafted. Some embodiments include human scaffold components for humanized antibodies. In one embodiment, the scaffold structure is a traditional, tetrameric antibody structure. Thus, embodiments of the IL-17RA-IL-17RC antagonist antibodies may include the additional components such as framework, J and D regions, constant regions, etc. that make up a heavy or light chain. Embodiments of the IL-17RA-IL-17RC antagonist antibodies may comprise antibodies that have a modified Fc domain, referred to as an Fc variant. An “Fc variant” refers to a molecule or sequence that is modified from a native Fc but still comprises a binding site for the salvage receptor, FcRn. Other examples of an “Fc variant” includes a molecule or sequence that is humanized from a non-human native Fc. Furthermore, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

Embodiments of IL-17RA-IL-17RC antagonist antibodies comprise human monoclonal antibodies. Human monoclonal antibodies directed against human IL-17RA, or IL-17RC, or both IL-17RA and IL-17RC may be made using any known methods known in the art, such as but not limited to XenoMouse™ technology (see, for example U.S. Pat. Nos. 6,114,598; 6,162,963; 6,833,268; 7,049,426; 7,064,244; Green et al, 1994, Nature Genetics 7:13-21; Mendez et al., 1997, Nature Genetics 15:146-156; Green and Jakobovitis, 1998, J. Ex. Med. 188:483-495). Other examples of making fully human antibodies include UltiMab Human Antibody Development System™ and Trans-Phage Technology™ (Medarex Corp., Princeton, N.J.), phage-display technologies, ribosome-display technologies (see for example Cambridge Antibody Technology, Cambridge, UK), as well as any other method known in the art.

Certain embodiments of IL-17RA-IL-17RC antagonist antibodies comprise chimeric and humanized antibodies, or fragments thereof. In general, both chimeric antibodies and humanized antibodies refer to antibodies that combine regions from more than one species. For example, chimeric antibodies traditionally comprise variable region(s) from a non-human species and the constant region(s) from a human. Humanized antibodies generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is well known in the art (see, for example Jones, 1986, Nature 321:522-525; Verhoeyen et al., 1988, Science 239:1534-1536). Humanized antibodies can also be generated using mice with a genetically engineered immune system or by any other method or technology known in the art (see for example Roque, at al., 2004, Biotechnol. Prog. 20:639-654). In some embodiments, the CDRs are human, and thus both humanized and chimeric antibodies in this context can include some non-human CDRs; for example, humanized antibodies may be generated that comprise the CDRH3 and CDRL3 regions, with one or more of the other CDR regions being of a different special origin.

In one embodiment, the IL-17RA-IL-17RC antagonist antibodies comprise a multispecific antibody. These are antibodies that bind to two (or more) different antigens. An example of a bispecific antibody known in the art are “diabodies”. Diabodies can be manufactured in a variety of ways known in the art, e.g., prepared chemically or from hybrid hybridomas (Holliger and Winter, 1993, Current Opinion Biotechnol. 4:446-449). A specific embodiment of a multispecific IL-17RA-IL-17RC antagonist antibody is an antibody that has the capacity to bind to both IL-17RA and IL-17RC.

In alternative embodiments, the IL-17RA-IL-17RC antagonist antibodies comprise a minibody. Minibodies are minimized antibody-like proteins comprising a scFv joined to a CH3 domain (see, for example Hu, et al., 1996, Cancer Res. 56:3055-3061).

In alternative embodiments, the IL-17RA-IL-17RC antagonist antibodies comprise a domain antibody; for example those described in U.S. Pat. No. 6,248,516. Domain antibodies (dAbs) are functional binding domains of antibodies, corresponding to the variable regions of either the heavy (VH) or light (VL) chains of human antibodies. dAbs have a molecular weight of approximately 13 kDa, or less than one-tenth the size of a full antibody. dAbs are well expressed in a variety of hosts including bacterial, yeast, and mammalian cell systems. In addition, dAbs are highly stable and retain activity even after being subjected to harsh conditions, such as freeze-drying or heat denaturation. See, for example, U.S. Pat. Nos. 6,291,158; 6,582,915; 6,593,081; 6,172,197; US Serial No. 2004/0110941; European Patent 0368684; U.S. Pat. No. 6,696,245, WO04/058821, WO04/003019 and WO03/002609.

As mentioned previously, the IL-17RA-IL-17RC antagonist antibodies may comprise an antibody fragment, i.e., a fragment of any of the antibodies mentioned herein that retain binding specificity to IL-17RA, or IL-17RC, or both IL-17RA and IL-17RC. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (see for example Ward, et al., 1989, Nature 341:544-546) which consists of a single variable, (v) isolated CDR regions, (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (see, for example Bird, et al., 1988 Science 242:423-426; Huston, et al., 1988, Proc. Natl. Acad. Sci. 85:5879-5883), (viii) bispecific single chain Fv dimers, and (ix) “diabodies” or “triabodies”, multivalent or multispecific fragments constructed by gene fusion (see, for example, Tomlinson, et. al., 2000, Methods Enzymol. 326:461-479; WO94/13804; Holliger, et al., 1993, Proc. Natl. Acad. Sci. 90:6444-6448). The antibody fragments may be modified. For example, the molecules may be stabilized by the incorporation of disulphide bridges linking the VH and VL domains (see, for example, Reiter, et al., 1996, Nature Biotech. 14:1239-1245). Again, as outlined herein, the non-CDR components of these fragments are preferably human sequences.

In further embodiments, the IL-17RA-IL-17RC antagonist antibodies comprise an antibody fusion protein (sometimes referred to herein as an “antibody conjugate”). The conjugate partner can be proteinaceous or non-proteinaceous; the latter generally being generated using functional groups on the antigen binding protein (see the discussion on covalent modifications of the antigen binding proteins) and on the conjugate partner. For example linkers are known in the art; for example, homo- or hetero-bifunctional linkers as are well known (see, for example, 1994 Pierce Chemical Company catalog, technical section on cross-linkers, pages 155-200, incorporated herein by reference). Suitable conjugates include, but are not limited to, labels as described below, drugs and cytotoxic agents including, but not limited to, cytotoxic drugs (e.g., chemotherapeutic agents) or toxins or active fragments of such toxins. Suitable toxins and their corresponding fragments include diptheria A chain, exotoxin A chain, ricin A chain, abrin A chain, curcin, crotin, phenomycin, enomycin and the like. Cytotoxic agents also include radiochemicals made by conjugating radioisotopes to antigen binding proteins, or binding of a radionuclide to a chelating agent that has been covalently attached to the antigen binding protein. Additional embodiments utilize calicheamicin, auristatins, geldanamycin and maytansine.

In one embodiment, the IL-17RA-IL-17RC antagonist antibodies comprise an antibody analog, sometimes referred to as “synthetic antibodies.” For example, a variety of alternative protein scaffolds or artificial scaffolds may be grafted with CDRs from IL-17RA-IL-17RC antagonist antibodies. Such scaffolds include, but are not limited to, mutations introduced to stabilize the three-dimensional structure of the binding protein as well as wholly synthetic scaffolds consisting for example of biocompatible polymers. See, for example, Korndorfer, et al., 2003, Proteins: Structure, Function, and Bioinformatics, Volume 53, Issue 1:121-129; Roque, et al., 2004, Biotechnol. Prog. 20:639-654. In alternative embodiments the IL-17RA-IL-17RC antagonist antibodies may comprise peptide antibody mimetics, or “PAMs”, as well as antibody mimetics utilizing fibronection components as a scaffold.

1.2 IL-17RA-IL-17RC Antagonists: Peptides/Polypeptides

Embodiments of IL-17RA-IL-17RC antagonists comprise proteins in the form of peptides and polypeptides that specifically bind to IL-17RA, or IL-17RC, or both IL-17RA and IL-17RC that inhibit the association of IL-17RA and IL-17RC in forming an IL-17RA-IL-17RC heteromeric receptor complex. Embodiments include recombinant IL-17RA-IL-17RC antagonists. A “recombinant protein” is a protein made using recombinant techniques, i.e., through the expression of a recombinant nucleic acid using methods known in the art.

A “peptide,” as used herein refers to molecules of 1 to 100 amino acids. Exemplary peptides that bind to IL-17RA, or IL-17RC, or both IL-17RA and IL-17RC that inhibit the association of IL-17RA and IL-17RC in forming an IL-17RA-IL-17RC heteromeric receptor complex or inhibit IL-17RA-IL-17RC heteromeric receptor complex signaling may comprise those generated from randomized libraries. For example, peptide sequences from fully random sequences (e.g., selected by phage display methods or RNA-peptide screening) and sequences in which one or more residues of a naturally occurring molecule is replaced by an amino acid residue not appearing in that position in the naturally occurring molecule. Exemplary methods for identifying peptide sequences include phage display, E. coli display, ribosome display, RNA-peptide screening, chemical screening, and the like.

By “protein,” as used herein, is meant at least two covalently attached amino acids, which includes proteins, polypeptides, oligopeptides and peptides. In some embodiments, the two or more covalently attached amino acids are attached by a peptide bond. The protein may be made up of naturally occurring amino acids and peptide bonds, for example when the protein is made recombinantly using expression systems and host cells, as outlined below. Alternatively, in some embodiments (for example when proteinaceous candidate agents are screened for the ability to inhibit IL-17RA and IL-17RC association) the protein may include synthetic amino acids (e.g., homophenylalanine, citrulline, ornithine, and norleucine), or peptidomimetic structures, i.e., “peptide or protein analogs”, such as peptoids (see, Simon et al., 1992, Proc. Natl. Acad. Sci. U.S.A. 89:9367, incorporated by reference herein), which can be resistant to proteases or other physiological and/or storage conditions. Such synthetic amino acids may be incorporated in particular when the antigen binding protein is synthesized in vitro by conventional methods well known in the art. In addition, any combination of peptidomimetic, synthetic and naturally occurring residues/structures can be used. “Amino acid” also includes imino acid residues such as proline and hydroxyproline. The amino acid “R group” or “side chain” may be in either the (L)- or the (S)-configuration. In a specific embodiment, the amino acids are in the (L)- or (S)-configuration.

In some embodiments, the antigen binding proteins of the invention are isolated proteins or substantially pure proteins. An “isolated” protein is unaccompanied by at least some of the material with which it is normally associated in its natural state, preferably constituting at least about 5%, more preferably at least about 50% by weight of the total protein in a given sample. A “substantially pure” protein comprises at least about 75% by weight of the total protein, with at least about 80% being specific, and at least about 90% being particularly specific. The definition includes the production of an antigen binding protein from one organism in a different organism or host cell. Alternatively, the protein may be made at a significantly higher concentration than is normally seen, through the use of an inducible promoter or high expression promoter, such that the protein is made at increased concentration levels.

2.0 IL-17RA-IL-17RC Antigen Binding Proteins: Modifications

As mentioned above, IL-17RA-IL-17RC antigen binding proteins include IL-17RA-IL-17RC antagonists, which includes, but is not limited to, antibodies, peptides, and polypeptides. Alternative embodiments of IL-17RA-IL-17RC antigen binding proteins (e.g., IL-17RA-IL-17RC antagonists) comprise covalent modifications of IL-17RA-IL-17RC antigen binding proteins. Such modifications may be done post-translationally. For example, several types of covalent modifications of the IL-17RA-IL-17RC antigen binding proteins are introduced into the molecule by reacting specific amino acid residues of the antigen binding protein with an organic derivatizing agent that is capable of reacting with selected side chains or the N- or C-terminal residues. The following represent examples of such modifications to the IL-17RA-IL-17RC antigen binding proteins.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in IL-17RAdioimmunoassay, the chloramine T method described above being suitable. Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking IL-17RA-IL-17RC antagonists to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention. Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983]), acetylation of the N-terminal amine, and amidation of any C-terminal carboxyl group.

Another type of covalent modification of the IL-17RA-IL-17RC antagonists included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used. Addition of glycosylation sites to the IL-17RA-IL-17RC antagonists is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antigen binding protein amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the IL-17RA-IL-17RC antagonists is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published Sep. 11, 1987, and in Aplin and Wriston, 1981, CRC Crit. Rev. Biochem., pp. 259-306.

Removal of carbohydrate moieties present on the starting IL-17RA-IL-17RC antagonists may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Another type of covalent modification of the IL-17RA-IL-17RC antagonists comprises linking the antigen binding protein to various nonproteinaceous polymers, including, but not limited to, various polyols such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antigen binding protein to facilitate the addition of polymers such as PEG.

In some embodiments, the covalent modification of the IL-17RA-IL-17RC antagonists of the invention comprises the addition of one or more labels. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antigen binding protein via spacer arms of various lengths to reduce potential steric hindrance. Various methods for labelling proteins are known in the art and may be used in performing the present invention. Specific labels include optical dyes, including, but not limited to, chromophores, phosphors and fluorophores, with the latter being specific in many instances. Fluorophores can be either “small molecule” fluores, or proteinaceous fluores.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, hereby expressly incorporated by reference.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

Covalent modifications of IL-17RA-IL-17RC antagonists are included within the scope of this invention, and are generally, but not always, done post-translationally. For example, several types of covalent modifications of the IL-17RA-IL-17RC antagonists are introduced into the molecule by reacting specific amino acid residues of the IL-17RA-IL-17RC antagonists with an organic derivatizing agent that is capable of reacting with selected side chains or the N- or C-terminal residues.

In some embodiments, the covalent modification of the antigen binding proteins of the invention comprises the addition of one or more labels. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antigen binding protein via spacer arms of various lengths to reduce potential steric hindrance. Various methods for labelling proteins are known in the art and may be used in performing the present invention.

Specific labels include optical dyes, including, but not limited to, chromophores, phosphors and fluorophores, with the latter being specific in many instances. Fluorophores can be either “small molecule” fluores, or proteinaceous fluores. By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, hereby expressly incorporated by reference.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), 13 galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

3.0 Methods of Use

Additional embodiments include methods of inhibiting IL-17RA and/or IL-17RC activation in cells expressing IL-17RA and IL-17RC using one or more of the IL-17RA-IL-17RC antagonists described herein. For example, a method of inhibiting IL-17RA and/or IL-17RC activation in cells expressing IL-17RA and IL-17RC comprises exposing said cells to an IL-17RA-IL-17RC antagonist, wherein the IL-17RA-IL-17RC antagonist binds IL-17RA and partially inhibits or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F. In one embodiment, the IL-17RA-IL-17RC antagonist need not block the binding of IL-17A and/or IL-17F from binding to IL-17RA. In alternative embodiments, the IL-17RA-IL-17RC antagonist may block the binding of IL-17A and/or IL-17F to IL-17RA. Additional embodiments comprise a method wherein said IL-17RA-IL-17RC antagonist is an antibody, as defined herein.

Additional embodiments include methods of inhibiting IL-17RA and/or IL-17RC activation in cells expressing IL-17RA and IL-17RC using one or more of the IL-17RA-IL-17RC antagonists described herein. For example, a method of inhibiting IL-17RA and/or IL-17RC activation in cells expressing IL-17RA and IL-17RC comprises exposing said cells to an IL-17RA-IL-17RC antagonist, wherein the IL-17RA-IL-17RC antagonist binds IL-17RC and partially inhibits or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F. In one embodiment, the IL-17RA-IL-17RC antagonist need not block the binding of IL-17A and/or IL-17F from binding to IL-17RC. In alternative embodiments, the IL IL-17RA-IL-17RC antagonist may block the binding of IL-17A and/or IL-17F to IL-17RC. Additional embodiments comprise a method wherein said IL-17RA-IL-17RC antagonist is an antibody, as defined herein. Additional embodiments include methods of inhibiting IL-17RA and/or IL-17RC activation in cells expressing IL-17RA and IL-17RC using one or more of the IL-17RA-IL-17RC antagonists described herein. For example, a method of inhibiting IL-17RA and/or IL-17RC activation in cells expressing IL-17RA and IL-17RC comprises exposing said cells to an IL-17RA-IL-17RC antagonist, wherein the IL-17RA-IL-17RC antagonist binds both IL-17RA and IL-17RC and partially inhibit or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F. In one embodiment, the IL-17RA-IL-17RC antagonist need not block the binding of IL-17A and/or IL-17F from binding to either IL-17RA or IL-17RC. In alternative embodiments, the IL-17RA-IL-17RC antagonist may block the binding of IL-17A and/or IL-17F to either IL-17RA or IL-17RC. Additional embodiments comprise a method wherein said IL-17RA-IL-17RC antagonist is an antibody, as defined herein.

Additional embodiments include methods of inhibiting IL-17RA and/or IL-17RC activation in cells expressing IL-17RA and IL-17RC in vivo using one or more of the IL-17RA-IL-17RC antagonists described herein. For example, a method of inhibiting IL-17RA and/or IL-17RC activation in cells expressing IL-17RA and IL-17RC in vivo comprises exposing said cells to an IL-17RA-IL-17RC antagonist, wherein the IL-17RA-IL-17RC antagonist binds IL-17RA and partially inhibits or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F. In one embodiment, the IL-17RA-IL-17RC antagonist need not block the binding of IL-17A and/or IL-17F from binding to IL-17RA. In alternative embodiments, the IL-17RA-IL-17RC antagonist may block the binding of IL-17A and/or IL-17F to IL-17RA. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments include methods of inhibiting IL-17RA and/or IL-17RC activation in cells expressing IL-17RA and IL-17RC in vivo using one or more of the IL-17RA-IL-17RC antagonists described herein. For example, a method of inhibiting IL-17RA and/or IL-17RC activation in cells expressing IL-17RA and IL-17RC in vivo comprises exposing said cells to an IL-17RA-IL-17RC antagonist, wherein the IL-17RA-IL-17RC antagonist binds IL-17RC and partially inhibits or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F. In one embodiment, the IL-17RA-IL-17RC antagonist need not block the binding of IL-17A and/or IL-17F from binding to IL-17RC. In alternative embodiments, the IL-17RA-IL-17RC antagonist may block the binding of IL-17A and/or IL-17F to IL-17RC. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments include methods of inhibiting IL-17RA and/or IL-17RC activation in cells expressing IL-17RA and IL-17RC in vivo using one or more of the IL-17RA-IL-17RC antagonists described herein. For example, a method of inhibiting IL-17RA and/or IL-17RC activation in cells expressing IL-17RA and IL-17RC in vivo comprises exposing said cells to an IL-17RA-IL-17RC antagonist, wherein the IL-17RA-IL-17RC antagonist binds both IL-17RA and IL-17RC and partially inhibit or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F. In one embodiment, the IL-17RA-IL-17RC antagonist need not block the binding of IL-17A and/or IL-17F from binding to either IL-17RA or IL-17RC. In alternative embodiments, the IL-17RA-IL-17RC antagonist may block the binding of IL-17A and/or IL-17F to either IL-17RA or IL-17RC. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments include methods of reducing proinflammatory mediators released after IL-17RA-IL-17RC heteromeric receptor complex activation in cells expressing said complex in vivo using one or more of the IL-17RA-IL-17RC antagonists described herein. For example, a method of reducing proinflammatory mediators released after IL-17RA-IL-17RC heteromeric receptor complex activation in cells expressing said complex in vivo comprises exposing said cells to an IL-17RA-IL-17RC antagonist, wherein the IL-17RA-IL-17RC antagonist binds IL-17RA and partially inhibits or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F, and consequent release of proinflammatory mediators. In one embodiment, the IL-17RA-IL-17RC antagonist need not block the binding of IL-17A and/or IL-17F from binding to IL-17RA. In alternative embodiments, the IL-17RA-IL-17RC antagonist may block the binding of IL-17A and/or IL-17F to IL-17RA. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments include methods of reducing proinflammatory mediators released after IL-17RA-IL-17RC heteromeric receptor complex activation in cells expressing said complex in vivo using one or more of the IL-17RA-IL-17RC antagonists described herein. For example, a method of reducing proinflammatory mediators released after IL-17RA-IL-17RC heteromeric receptor complex activation in cells expressing said complex in vivo comprises exposing said cells to an IL-17RA-IL-17RC antagonist, wherein the IL-17RA-IL-17RC antagonist binds IL-17RC and partially inhibits or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F, and consequent release of proinflammatory mediators. In one embodiment, the IL-17RA-IL-17RC antagonist need not block the binding of IL-17A and/or IL-17F from binding to IL-17RC. In alternative embodiments, the IL-17RA-IL-17RC antagonist may block the binding of IL-17A and/or IL-17F to IL-17RC. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments include methods of reducing proinflammatory mediators released after IL-17RA-IL-17RC heteromeric receptor complex activation in cells expressing said complex in vivo using one or more of the IL-17RA-IL-17RC antagonists described herein. For example, a method of reducing proinflammatory mediators released after IL-17RA-IL-17RC heteromeric receptor complex activation in cells expressing said complex in vivo comprises exposing said cells to an IL-17RA-IL-17RC antagonist, wherein the IL-17RA-IL-17RC antagonist binds both IL-17RA and IL-17RC and partially inhibit or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F, and consequent release of proinflammatory mediators. In one embodiment, the IL-17RA-IL-17RC antagonist need not block the binding of IL-17A and/or IL-17F from binding to either IL-17RA or IL-17RC. In alternative embodiments, the IL-17RA-IL-17RC antagonist may block the binding of IL-17A and/or IL-17F to either IL-17RA or IL-17RC. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments comprise methods, as described above, wherein the proinflammatory mediator is at least one of the following: IL-6, IL-8, CXCL1, CXCL2, GM-CSF, G-CSF, M-CSF, IL-1β, TNFα, RANK-L, LIF, PGE2, IL-12, MMP3, MMP9, GROα, NO, as well as any other proinflammatory mediator known in the art to be released from any cells through activation of IL-17RA and/or IL-17RC.

Further embodiments include methods of treating IL-17 family member-associated disorders, such as but not limited to, inflammatory and autoimmune disorders with the IL-17RA-IL-17RC antagonists.

Additional embodiments include methods of treating inflammation, wherein the IL-17RA-IL-17RC heteromeric receptor complex is partially or fully blocked from being activated by administering one or more of the IL-17RA-IL-17RC antagonists described herein. For example, a method of treating inflammation in a patient in need thereof comprises administering to said patient an IL-17RA-IL-17RC antagonist, wherein the IL-17RA-IL-17RC antagonist binds IL-17RA and partially inhibits or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F, and consequent release of proinflammatory mediators. In one embodiment, the IL-17RA-IL-17RC antagonist need not block the binding of IL-17A and/or IL-17F from binding to IL-17RA. In alternative embodiments, the IL-17RA-IL-17RC antagonist may block the binding of IL-17A and/or IL-17F to IL-17RA. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments include methods of treating inflammation, wherein the IL-17RA-IL-17RC heteromeric receptor complex is partially or fully blocked from being activated by administering one or more of the IL-17RA-IL-17RC antagonists described herein. For example, a method of treating inflammation in a patient in need thereof comprises administering to said patient an IL-17RA-IL-17RC antagonist, wherein the IL-17RA-IL-17RC antagonist binds IL-17RC and partially inhibits or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F, and consequent release of proinflammatory mediators. In one embodiment, the IL-17RA-IL-17RC antagonist need not block the binding of IL-17A and/or IL-17F from binding to IL-17RC. In alternative embodiments, the IL-17RA-IL-17RC antagonist may block the binding of IL-17A and/or IL-17F to IL-17RC. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments include methods of treating inflammation, wherein the IL-17RA-IL-17RC heteromeric receptor complex is partially or fully blocked from being activated by administering one or more of the IL-17RA-IL-17RC antagonists described herein. For example, a method of treating inflammation in a patient in need thereof comprises administering to said patient an IL-17RA-IL-17RC antagonist, wherein the IL-17RA-IL-17RC antagonist binds both IL-17RA and IL-17RC and partially inhibit or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F, and consequent release of proinflammatory mediators. In one embodiment, the IL-17RA-IL-17RC antagonist need not block the binding of IL-17A and/or IL-17F from binding to either IL-17RA or IL-17RC. In alternative embodiments, the IL-17RA-IL-17RC antagonist may block the binding of IL-17A and/or IL-17F to either IL-17RA or IL-17RC. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments include methods of treating an autoimmune disorder, wherein the IL-17RA-IL-17RC heteromeric receptor complex is partially or fully blocked from being activated by administering one or more of the IL-17RA-IL-17RC antagonists described herein. For example, a method of treating an autoimmune disorder in a patient in need thereof comprises administering to said patient an IL-17RA-IL-17RC antagonist, wherein the IL-17RA-IL-17RC antagonist binds IL-17RA and partially inhibits or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F, and consequent release of proinflammatory mediators.

In one embodiment, the IL-17RA-IL-17RC antagonist need not block the binding of IL-17A and/or IL-17F from binding to IL-17RA. In alternative embodiments, the IL-17RA-IL-17RC antagonist may block the binding of IL-17A and/or IL-17F to IL-17RA. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments include methods of treating an autoimmune disorder, wherein the IL-17RA-IL-17RC heteromeric receptor complex is partially or fully blocked from being activated by administering one or more of the IL-17RA-IL-17RC antagonists described herein. For example, a method of treating an autoimmune disorder in a patient in need thereof comprises administering to said patient an IL-17RA-IL-17RC antagonist, wherein the IL-17RA-IL-17RC antagonist binds IL-17RC and partially inhibits or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F, and consequent release of proinflammatory mediators. In one embodiment, the IL-17RA-IL-17RC antagonist need not block the binding of IL-17A and/or IL-17F from binding to IL-17RC. In alternative embodiments, the IL-17RA-IL-17RC antagonist may block the binding of IL-17A and/or IL-17F to IL-17RC. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Additional embodiments include methods of treating an autoimmune disorder, wherein the IL-17RA-IL-17RC heteromeric receptor complex is partially or fully blocked from being activated by administering one or more of the IL-17RA-IL-17RC antagonists described herein. For example, a method of treating an autoimmune disorder in a patient in need thereof comprises administering to said patient an IL-17RA-IL-17RC antagonist, wherein the IL-17RA-IL-17RC antagonist binds both IL-17RA and IL-17RC and partially inhibit or fully inhibits association of IL-17RC with IL-17RA and thereby preventing IL-17RA-IL-17RC heteromeric receptor complex formation and activation through binding of IL-17 ligand family members, such as but not limited to IL-17A and IL-17F, and consequent release of proinflammatory mediators. In one embodiment, the IL-17RA-IL-17RC antagonist need not block the binding of IL-17A and/or IL-17F from binding to either IL-17RA or IL-17RC. In alternative embodiments, the IL-17RA-IL-17RC antagonist may block the binding of IL-17A and/or IL-17F to either IL-17RA or IL-17RC. Additional embodiments comprise a method wherein said IL-17RA-IL-17RC antagonist is an antibody, as defined herein. Additional embodiments comprise a method wherein said IL-17RA-IL-17RC antagonist is an antibody, as defined herein, and the antibody is in the form of a pharmaceutical composition.

Further embodiments include methods of treating inflammation and autoimmune disorders, as described above, wherein the autoimmune disorders include, but are not limited to, cartilage inflammation, and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, Graves' disease, Addison's disease, Raynaud's phenomenon, autoimmune hepatitis, GVHD, and the like.

Additional embodiments include pharmaceutical compositions comprising a therapeutically effective amount of one or more of an IL-17RA-IL-17RC antagonist together with a pharmaceutically acceptable diluent, carrier, solubilizer, emulsifier, preservative, and/or adjuvant. In addition, the invention provides methods of treating a patient by administering such pharmaceutical composition. Acceptable formulation materials are nontoxic to recipients at the dosages and concentrations employed. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the IL-17RA-IL-17RC antagonist. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute. In certain embodiments, IL-17RA-IL-17RC antagonist compositions may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the IL-17RA-IL-17RC antagonist product may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the IL-17RA-IL-17RC antagonists may be provided in the form of a pyrogen-free, parenterally acceptable aqueous solution comprising the desired IL-17 receptor antigen binding protein in a pharmaceutically acceptable vehicle. A particularly suitable vehicle for parenteral injection is sterile distilled water in which the IL-17RA-IL-17RC antagonist is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen binding protein.

Pharmaceutical compositions of the invention can be formulated for inhalation. In these embodiments, IL-17RA-IL-17RC antagonist may be formulated as a dry, inhalable powder. Inhalation solutions may also be formulated with a propellant for aerosol delivery. In certain embodiments, solutions may be nebulized. Pulmonary administration and formulation methods therefore are further described in International Patent Application No. PCT/US94/001875, which is incorporated by reference and describes pulmonary delivery of chemically modified proteins.

It is also contemplated that formulations can be administered orally. IL-17RA-IL-17RC antagonists that are administered in this fashion can be formulated with or without carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. Additional agents can be included to facilitate absorption of the IL-17RA-IL-17RC antagonist. Diluents, flavorings, low melting point waxes, vegetable oils, lubricants, suspending agents, tablet disintegrating agents, and binders may also be employed.

A pharmaceutical composition of the invention is preferably provided to comprise an effective quantity of one or more IL-17RA-IL-17RC antagonists in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving IL-17RA-IL-17RC antagonists in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP 058481, each of which is incorporated by reference), copolymers of L-glutamic acid and gamma ethyl-L-glutamate (Sidman et al., 1983, Biopolymers 2:547-556), poly (2-hydroxyethyl-inethacrylate) (Langer, et al., 1981, J. Biomed. Mater. Res. 15:167-277 and Langer, 1982, Chem. Tech. 12:98-105), ethylene vinyl acetate (Langer, et al., 1981, supra) or poly-D(−)-3-hydroxybutyric acid (European Patent Application Publication No. EP 133,988). Sustained release compositions may also include liposomes that can be prepared by any of several methods known in the art. See, e.g., Eppstein, et al., 1985, Proc. Natl. Acad. Sci. U.S.A. 82:3688-3692; European Patent Application Publication Nos. EP 036,676; EP 088,046 and EP 143,949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the invention may each contain both a first container having a dried protein and a second container having an aqueous formulation. In certain embodiments of this invention, kits containing single and multi-chambered pre-filled syringes (e.g., liquid syringes and lyosyringes) are provided.

The therapeutically effective amount of an IL-17RA-IL-17RC antagonist-containing pharmaceutical composition to be employed will depend, for example, upon the therapeutic context and objectives. One skilled in the art will appreciate that the appropriate dosage levels for treatment will vary depending, in part, upon the molecule delivered, the indication for which the IL-17RA-IL-17RC antagonist is being used, the route of administration, and the size (body weight, body surface or organ size) and/or condition (the age and general health) of the patient. In certain embodiments, the clinician may titer the dosage and modify the route of administration to obtain the optimal therapeutic effect. A typical dosage may range from about 0.1 μg/kg to up to about 30 mg/kg or more, depending on the factors mentioned above. In specific embodiments, the dosage may range from 0.1 μg/kg up to about 30 mg/kg, optionally from 1 μg/kg up to about 30 mg/kg or from 10 μg/kg up to about 5 mg/kg. Of course, it is understood that this is to be determined by qualified physicians and that these doses are merely exemplary. Dosing frequency will depend upon the pharmacokinetic parameters of the particular IL-17RA-IL-17RC antagonist in the formulation used. Typically, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In certain embodiments, the IL-17RA-IL-17RC antagonists can be administered to patients throughout an extended time period. Chronic administration of an IL-17RA-IL-17RC antagonist may minimize the adverse immune or allergic response commonly associated with IL-17RA-IL-17RC antagonist that are not fully human, for example an antibody raised against a human antigen in a non-human animal, for example, a non-fully human antibody or non-human antibody produced in a non-human species.

The route of administration of the pharmaceutical composition is in accord with known methods, e.g., orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

The composition also may be administered locally via implantation of a membrane, sponge or another appropriate material onto which the desired molecule has been absorbed or encapsulated. In certain embodiments, where an implantation device is used, the device may be implanted into any suitable tissue or organ, and delivery of the desired molecule may be via diffusion, timed-release bolus, or continuous administration.

The IL-17RA-IL-17RC antagonists described herein may be used in combination (pre-treatment, post-treatment, or concurrent treatment) with any of one or more TNF inhibitors for the treatment or prevention of the diseases and disorders recited herein, such as but not limited to, all forms of soluble TNF receptors including Etanercept (such as ENBREL®), as well as all forms of monomeric or multimeric p75 and/or p55 TNF receptor molecules and fragments thereof; anti-human TNF antibodies, such as but not limited to, Infliximab (such as REMICADE®), and D2E7 (such as HUMIRA®), and the like. Such TNF inhibitors include compounds and proteins which block in vivo synthesis or extracellular release of TNF. In a specific embodiment, the present invention is directed to the use of an IL-17RA-IL-17RC antagonist in combination (pre-treatment, post-treatment, or concurrent treatment) with any of one or more of the following TNF inhibitors: TNF binding proteins (soluble TNF receptor type-I and soluble TNF receptor type-II (“sTNFRs”), as defined herein), anti-TNF antibodies, granulocyte colony stimulating factor; thalidomide; BN 50730; tenidap; E 5531; tiapafant PCA 4248; nimesulide; panavir; rolipram; RP 73401; peptide T; MDL 201,449A; (1R,3S)-Cis-1-[9-(2,6-diaminopurinyl)]-3-hydroxy-4-cyclopentene hydrochloride; (1R,3R)-trans-1-(9-(2,6-diamino)purine]-3-acetoxycyclopentane; (1R,3R)-trans-1-[9-adenyl)-3-azidocyclopentane hydrochloride and (1R,3R)-trans-1-(6-hydroxy-purin-9-yl)-3-azidocyclo-pentane. TNF binding proteins are disclosed in the art (EP 308 378, EP 422 339, GB 2 218 101, EP 393 438, WO 90/13575, EP 398 327, EP 412 486, WO 91/03553, EP 418 014, JP 127,800/1991, EP 433 900, U.S. Pat. No. 5,136,021, GB 2 246 569, EP 464 533, WO 92/01002, WO 92/13095, WO 92/16221, EP 512 528, EP 526 905, WO 93/07863, EP 568 928, WO 93/21946, WO 93/19777, EP 417 563, WO 94/06476, and PCT International Application No. PCT/US97/12244). For example, EP 393 438 and EP 422 339 teach the amino acid and nucleic acid sequences of a soluble TNF receptor type I (also known as “sTNFR-I” or “30 kDa TNF inhibitor”) and a soluble TNF receptor type II (also known as “sTNFR-II” or “40 kDa TNF inhibitor”), collectively termed “sTNFRs”, as well as modified forms thereof (e.g., fragments, functional derivatives and variants). EP 393 438 and EP 422 339 also disclose methods for isolating the genes responsible for coding the inhibitors, cloning the gene in suitable vectors and cell types and expressing the gene to produce the inhibitors. Additionally, polyvalent forms (i.e., molecules comprising more than one active moiety) of sTNFR-I and sTNFR-II have also been disclosed. In one embodiment, the polyvalent form may be constructed by chemically coupling at least one TNF inhibitor and another moiety with any clinically acceptable linker, for example polyethylene glycol (WO 92/16221 and WO 95/34326), by a peptide linker (Neve et al. (1996), Cytokine, 8(5):365-370, by chemically coupling to biotin and then binding to avidin (WO 91/03553) and, finally, by combining chimeric antibody molecules (U.S. Pat. No. 5,116,964, WO 89/09622, WO 91/16437 and EP 315062. Anti-TNF antibodies include the MAK 195F Fab antibody (Holler et al. (1993), 1st International Symposium on Cytokines in Bone Marrow Transplantation, 147); CDP 571 anti-TNF monoclonal antibody (Rankin et al. (1995), British Journal of Rheumatology, 34:334-342); BAY X 1351 murine anti-tumor necrosis factor monoclonal antibody (Kieft et al. (1995), 7th European Congress of Clinical Microbiology and Infectious Diseases, page 9); CenTNF cA2 anti-TNF monoclonal antibody (Elliott et al. (1994), Lancet, 344:1125-1127 and Elliott et al. (1994), Lancet, 344:1105-1110).

The IL-17RA-IL-17RC antagonists described herein may be used in combination with all forms of IL-1 inhibitors, such as but not limited to, kiniret (for example ANAKINRA®) (pretreatment, post-treatment, or concurrent treatment). Interleukin-1 receptor antagonist (IL-1ra) is a human protein that acts as a natural inhibitor of interleukin-1. Interleukin-1 receptor antagonists, as well as the methods of making and methods of using thereof, are described in U.S. Pat. No. 5,075,222; WO 91/08285; WO 91/17184; AU 9173636; WO 92/16221; WO 93/21946; WO 94/06457; WO 94/21275; FR 2706772; WO 94/21235; DE 4219626; WO 94/20517; WO 96/22793 and WO 97/28828. The proteins include glycosylated as well as non-glycosylated IL-1 receptor antagonists. Specifically, three preferred forms of IL-1ra (IL-1raα, IL-1raβ and IL-1rax), each being encoded by the same DNA coding sequence and variants thereof, are disclosed and described in U.S. Pat. No. 5,075,222. Methods for producing IL-1 inhibitors, particularly IL-1ras, are also disclosed in the U.S. Pat. No. 5,075,222. An additional class of interleukin-1 inhibitors includes compounds capable of specifically preventing activation of cellular receptors to IL-1. Such compounds include IL-1 binding proteins, such as soluble receptors and monoclonal antibodies. Such compounds also include monoclonal antibodies to the receptors. A further class of interleukin-1 inhibitors includes compounds and proteins that block in vivo synthesis and/or extracellular release of IL-1. Such compounds include agents that affect transcription of IL-1 genes or processing of IL-1 preproteins.

The IL-17RA-IL-17RC antagonists described herein may be used in combination with all forms of CD28 inhibitors, such as but not limited to, abatacept (for example ORENCIA®) (pretreatment, post-treatment, or concurrent treatment). The IL-17RA-IL-17RC antagonists may be used in combination with one or more cytokines, lymphokines, hematopoietic factor(s), and/or an anti-inflammatory agent (pretreatment, post-treatment, or concurrent treatment).

Treatment of the diseases and disorders recited herein can include the use of first line drugs for control of pain and inflammation in combination (pretreatment, post-treatment, or concurrent treatment) with treatment with one or more of the IL-17RA-IL-17RC antagonists provided herein. These drugs are classified as non-steroidal, anti-inflammatory drugs (NSAIDs). Secondary treatments include corticosteroids, slow acting antirheumatic drugs (SAARDs), or disease modifying (DM) drugs. Information regarding the following compounds can be found in The Merck Manual of Diagnosis and Therapy, Sixteenth Edition, Merck, Sharp & Dohme Research Laboratories, Merck & Co., Rahway, N.J. (1992) and in Pharmaprojects, PJB Publications Ltd.

In a specific embodiment, the present invention is directed to the use of an IL-17RA-IL-17RC antagonist and any of one or more NSAIDs for the treatment of the diseases and disorders recited herein (pretreatment, post-treatment, or concurrent treatment). NSAIDs owe their anti-inflammatory action, at least in part, to the inhibition of prostaglandin synthesis (Goodman and Gilman in “The Pharmacological Basis of Therapeutics,” MacMillan 7th Edition (1985)). NSAIDs can be characterized into at least nine groups: (1) salicylic acid derivatives; (2) propionic acid derivatives; (3) acetic acid derivatives; (4) fenamic acid derivatives; (5) carboxylic acid derivatives; (6) butyric acid derivatives; (7) oxicams; (8) pyrazoles and (9) pyrazolones.

In another specific embodiment, the present invention is directed to the use of an IL-17RA-IL-17RC antagonist in combination (pretreatment, post-treatment, or concurrent treatment) with any of one or more salicylic acid derivatives, prodrug esters or pharmaceutically acceptable salts thereof. Such salicylic acid derivatives, prodrug esters and pharmaceutically acceptable salts thereof comprise: acetaminosalol, aloxiprin, aspirin, benorylate, bromosaligenin, calcium acetylsalicylate, choline magnesium trisalicylate, magnesium salicylate, choline salicylate, diflusinal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide O-acetic acid, salsalate, sodium salicylate and sulfasalazine. Structurally related salicylic acid derivatives having similar analgesic and anti-inflammatory properties are also intended to be encompassed by this group.

In an additional specific embodiment, the present invention is directed to the use of IL-17RA-IL-17RC antagonist in combination (pretreatment, post-treatment, or concurrent treatment) with any of one or more propionic acid derivatives, prodrug esters or pharmaceutically acceptable salts thereof. The propionic acid derivatives, prodrug esters, and pharmaceutically acceptable salts thereof comprise: alminoprofen, benoxaprofen, bucloxic acid, carprofen, dexindoprofen, fenoprofen, flunoxaprofen, fluprofen, flurbiprofen, furcloprofen, ibuprofen, ibuprofen aluminum, ibuproxam, indoprofen, isoprofen, ketoprofen, loxoprofen, miroprofen, naproxen, naproxen sodium, oxaprozin, piketoprofen, pimeprofen, pirprofen, pranoprofen, protizinic acid, pyridoxiprofen, suprofen, tiaprofenic acid and tioxaprofen. Structurally related propionic acid derivatives having similar analgesic and anti-inflammatory properties are also intended to be encompassed by this group.

In yet another specific embodiment, the present invention is directed to the use of an IL-17RA-IL-17RC antagonist in combination (pretreatment, post-treatment, or concurrent treatment) with any of one or more acetic acid derivatives, prodrug esters or pharmaceutically acceptable salts thereof. The acetic acid derivatives, prodrug esters, and pharmaceutically acceptable salts thereof comprise: acemetacin, alclofenac, amfenac, bufexamac, cinmetacin, clopirac, delmetacin, diclofenac potassium, diclofenac sodium, etodolac, felbinac, fenclofenac, fenclorac, fenclozic acid, fentiazac, furofenac, glucametacin, ibufenac, indomethacin, isofezolac, isoxepac, lonazolac, metiazinic acid, oxametacin, oxpinac, pimetacin, proglumetacin, sulindac, talmetacin, tiaramide, tiopinac, tolmetin, tolmetin sodium, zidometacin and zomepirac. Structurally related acetic acid derivatives having similar analgesic and anti-inflammatory properties are also intended to be encompassed by this group.

In another specific embodiment, the present invention is directed to the use of an IL-17RA-IL-17RC antagonist in combination (pretreatment, post-treatment, or concurrent treatment) with any of one or more fenamic acid derivatives, prodrug esters or pharmaceutically acceptable salts thereof. The fenamic acid derivatives, prodrug esters and pharmaceutically acceptable salts thereof comprise: enfenamic acid, etofenamate, flufenamic acid, isonixin, meclofenamic acid, meclofenamate sodium, medofenamic acid, mefenamic acid, niflumic acid, talniflumate, terofenamate, tolfenamic acid and ufenamate. Structurally related fenamic acid derivatives having similar analgesic and anti-inflammatory properties are also intended to be encompassed by this group.

In an additional specific embodiment, the present invention is directed to the use of an IL-17RA-IL-17RC antagonist in combination (pretreatment, post-treatment, or concurrent treatment) with any of one or more carboxylic acid derivatives, prodrug esters or pharmaceutically acceptable salts thereof. The carboxylic acid derivatives, prodrug esters, and pharmaceutically acceptable salts thereof which can be used comprise: clidanac, diflunisal, flufenisal, inoridine, ketorolac and tinoridine. Structurally related carboxylic acid derivatives having similar analgesic and anti-inflammatory properties are also intended to be encompassed by this group.

In yet another specific embodiment, the present invention is directed to the use of an IL-17RA-IL-17RC antagonist in combination (pretreatment, post-treatment, or concurrent treatment) with any of one or more butyric acid derivatives, prodrug esters or pharmaceutically acceptable salts thereof. The butyric acid derivatives, prodrug esters, and pharmaceutically acceptable salts thereof comprise: bumadizon, butibufen, fenbufen and xenbucin. Structurally related butyric acid derivatives having similar analgesic and anti-inflammatory properties are also intended to be encompassed by this group.

In another specific embodiment, the present invention is directed to the use of an IL-17RA-IL-17RC antagonist in combination (pretreatment, post-treatment, or concurrent treatment) with any of one or more oxicams, prodrug esters, or pharmaceutically acceptable salts thereof. The oxicams, prodrug esters, and pharmaceutically acceptable salts thereof comprise: droxicam, enolicam, isoxicam, piroxicam, sudoxicam, tenoxicam and 4-hydroxyl-1,2-benzothiazine 1,1-dioxide 4-(N-phenyl)-carboxamide. Structurally related oxicams having similar analgesic and anti-inflammatory properties are also intended to be encompassed by this group.

In still another specific embodiment, the present invention is directed to the use of an IL-17RA-IL-17RC antagonist in combination (pretreatment, post-treatment, or concurrent treatment) with any of one or more pyrazoles, prodrug esters, or pharmaceutically acceptable salts thereof. The pyrazoles, prodrug esters, and pharmaceutically acceptable salts thereof which may be used comprise: difenamizole and epirizole. Structurally related pyrazoles having similar analgesic and anti-inflammatory properties are also intended to be encompassed by this group.

In an additional specific embodiment, the present invention is directed to the use of an IL-17RA-IL-17RC antagonist in combination (pretreatment, post-treatment or, concurrent treatment) with any of one or more pyrazolones, prodrug esters, or pharmaceutically acceptable salts thereof. The pyrazolones, prodrug esters and pharmaceutically acceptable salts thereof which may be used comprise: apazone, azapropazone, benzpiperylon, feprazone, mofebutazone, morazone, oxyphenbutazone, phenylbutazone, pipebuzone, propylphenazone, ramifenazone, suxibuzone and thiazolinobutazone. Structurally related pyrazalones having similar analgesic and anti-inflammatory properties are also intended to be encompassed by this group.

In another specific embodiment, the present invention is directed to the use of an IL-17RA-IL-17RC antagonist in combination (pretreatment, post-treatment, or concurrent treatment) with any of one or more of the following NSAIDs: ε-acetamidocaproic acid, S-adenosyl-methionine, 3-amino-4-hydroxybutyric acid, amixetrine, anitrazafen, antrafenine, bendazac, bendazac lysinate, benzydamine, beprozin, broperamole, bucolome, bufezolac, ciproquazone, cloximate, dazidamine, deboxamet, detomidine, difenpiramide, difenpyramide, difisalamine, ditazol, emorfazone, fanetizole mesylate, fenflumizole, floctafenine, flumizole, flunixin, fluproquazone, fopirtoline, fosfosal, guaimesal, guaiazolene, isonixirn, lefetamine HCl, leflunomide, lofemizole, lotifazole, lysin clonixinate, meseclazone, nabumetone, nictindole, nimesulide, orgotein, orpanoxin, oxaceprol, oxapadol, paranyline, perisoxal, perisoxal citrate, pifoxime, piproxen, pirazolac, pirfenidone, proquazone, proxazole, thielavin B, tiflamizole, timegadine, tolectin, tolpadol, tryptamid and those designated by company code number such as 480156S, AA861, AD1590, AFP802, AFP860, AI77B, AP504, AU8001, BPPC, BW540C, CHINOIN 127, CN100, EB382, EL508, F1044, FK-506, GV3658, ITF182, KCNTEI6090, KME4, LA2851, MR714, MR897, MY309, ONO3144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301 and WY41770. Structurally related NSAIDs having similar analgesic and anti-inflammatory properties to the NSAIDs are also intended to be encompassed by this group.

In still another specific embodiment, the present invention is directed to the use of an IL-17RA-IL-17RC antagonist in combination (pretreatment, post-treatment or concurrent treatment) with any of one or more corticosteroids, prodrug esters or pharmaceutically acceptable salts thereof for the treatment of the diseases and disorders recited herein. Corticosteroids, prodrug esters and pharmaceutically acceptable salts thereof include hydrocortisone and compounds which are derived from hydrocortisone, such as 21-acetoxypregnenolone, alclomerasone, algestone, amcinonide, beclomethasone, betamethasone, betamethasone valerate, budesonide, chloroprednisone, clobetasol, clobetasol propionate, clobetasone, clobetasone butyrate, clocortolone, cloprednol, corticosterone, cortisone, cortivazol, deflazacon, desonide, desoximerasone, dexamethasone, diflorasone, diflucortolone, difluprednate, enoxolone, fluazacort, flucloronide, flumethasone, flumethasone pivalate, flucinolone acetonide, flunisolide, fluocinonide, fluorocinolone acetonide, fluocortin butyl, fluocortolone, fluocortolone hexanoate, diflucortolone valerate, fluorometholone, fluperolone acetate, fluprednidene acetate, fluprednisolone, flurandenolide, formocortal, halcinonide, halometasone, halopredone acetate, hydro-cortamate, hydrocortisone, hydrocortisone acetate, hydro-cortisone butyrate, hydrocortisone phosphate, hydrocortisone 21-sodium succinate, hydrocortisone tebutate, mazipredone, medrysone, meprednisone, methylprednisolone, mometasone furoate, paramethasone, prednicarbate, prednisolone, prednisolone 21-diedryaminoacetate, prednisolone sodium phosphate, prednisolone sodium succinate, prednisolone sodium 21-m-sulfobenzoate, prednisolone sodium 21-stearoglycolate, prednisolone tebutate, prednisolone 21-trimethylacetate, prednisone, prednival, prednylidene, prednylidene 21-diethylaminoacetate, tixocortol, triamcinolone, triamcinolone acetonide, triamcinolone benetonide and triamcinolone hexacetonide. Structurally related corticosteroids having similar analgesic and anti-inflammatory properties are also intended to be encompassed by this group.

In another specific embodiment, the present invention is directed to the use of an IL-17RA-IL-17RC antagonist in combination (pretreatment, post-treatment, or concurrent treatment) with any of one or more slow-acting antirheumatic drugs (SAARDs) or disease modifying antirheumatic drugs (DMARDS), prodrug esters, or pharmaceutically acceptable salts thereof for the treatment of the diseases and disorders recited herein. SAARDs or DMARDS, prodrug esters and pharmaceutically acceptable salts thereof comprise: allocupreide sodium, auranofin, aurothioglucose, aurothioglycanide, azathioprine, brequinar sodium, bucillamine, calcium 3-aurothio-2-propanol-1-sulfonate, chlorambucil, chloroquine, clobuzarit, cuproxoline, cyclo-phosphamide, cyclosporin, dapsone, 15-deoxyspergualin, diacerein, glucosamine, gold salts (e.g., cycloquine gold salt, gold sodium thiomalate, gold sodium thiosulfate), hydroxychloroquine, hydroxychloroquine sulfate, hydroxyurea, kebuzone, levamisole, lobenzarit, melittin, 6-mercaptopurine, methotrexate, mizoribine, mycophenolate mofetil, myoral, nitrogen mustard, D-penicillamine, pyridinol imidazoles such as SKNF86002 and SB203580, rapamycin, thiols, thymopoietin and vincristine. Structurally related SAARDs or DMARDs having similar analgesic and anti-inflammatory properties are also intended to be encompassed by this group.

In another specific embodiment, the present invention is directed to the use of an IL-17RA-IL-17RC antagonist (pretreatment, post-treatment, or concurrent treatment) with any of one or more COX2 inhibitors, prodrug esters or pharmaceutically acceptable salts thereof for the treatment of the diseases and disorders recited herein. Examples of COX2 inhibitors, prodrug esters or pharmaceutically acceptable salts thereof include, for example, celecoxib. Structurally related COX2 inhibitors having similar analgesic and anti-inflammatory properties are also intended to be encompassed by this group. Examples of COX-2 selective inhibitors include but not limited to etoricoxib, valdecoxib, celecoxib, licofelone, lumiracoxib, rofecoxib, and the like.

In still another specific embodiment, the present invention is directed to the use of an IL-17RA-IL-17RC antagonist in combination (pretreatment, post-treatment, or concurrent treatment) with any of one or more antimicrobials, prodrug esters or pharmaceutically acceptable salts thereof for the treatment of the diseases and disorders recited herein. Antimicrobials include, for example, the broad classes of penicillins, cephalosporins and other beta-lactams, aminoglycosides, azoles, quinolones, macrolides, rifamycins, tetracyclines, sulfonamides, lincosamides and polymyxins. The penicillins include, but are not limited to penicillin G, penicillin V, methicillin, nafcillin, oxacillin, cloxacillin, dicloxacillin, floxacillin, ampicillin, ampicillin/sulbactam, amoxicillin, amoxicillin/clavulanate, hetacillin, cyclacillin, bacampicillin, carbenicillin, carbenicillin indanyl, ticarcillin, ticarcillin/clavulanate, azlocillin, mezlocillin, peperacillin, and mecillinam. The cephalosporins and other beta-lactams include, but are not limited to cephalothin, cephapirin, cephalexin, cephradine, cefazolin, cefadroxil, cefaclor, cefamandole, cefotetan, cefoxitin, ceruroxime, cefonicid, ceforadine, cefixime, cefotaxime, moxalactam, ceftizoxime, cetriaxone, cephoperazone, ceftazidime, imipenem and aztreonam. The aminoglycosides include, but are not limited to streptomycin, gentamicin, tobramycin, amikacin, netilmicin, kanamycin and neomycin. The azoles include, but are not limited to fluconazole. The quinolones include, but are not limited to nalidixic acid, norfloxacin, enoxacin, ciprofloxacin, ofloxacin, sparfloxacin and temafloxacin. The macrolides include, but are not limited to erythomycin, spiramycin and azithromycin. The rifamycins include, but are not limited to rifampin. The tetracyclines include, but are not limited to spicycline, chlortetracycline, clomocycline, demeclocycline, deoxycycline, guamecycline, lymecycline, meclocycline, methacycline, minocycline, oxytetracycline, penimepicycline, pipacycline, rolitetracycline, sancycline, senociclin and tetracycline. The sulfonamides include, but are not limited to sulfanilamide, sulfamethoxazole, sulfacetamide, sulfadiazine, sulfisoxazole and co-trimoxazole (trimethoprim/sulfamethoxazole). The lincosamides include, but are not limited to clindamycin and lincomycin. The polymyxins (polypeptides) include, but are not limited to polymyxin B and colistin.

4.0 Screening Assays

Additional embodiments include methods of screening for antagonists of the IL-17RA-IL-17RC heteromeric receptor complex. Screening assay formats that are known in the art and are adaptable to identifying antagonists of the IL-17RA-IL-17RC heteromeric receptor complex are contemplated. For example: a method of screening for an antagonist of an IL-17RA-IL-17RC heteromeric receptor complex, comprising providing an IL-17RA and an IL-17RC in an IL-17RA-IL-17RC heteromeric receptor complex; exposing a candidate agent to said receptor complex; and determining the amount of receptor complex formation relative to not having been exposed to the candidate agent. The step of exposing a candidate agent to the receptor complex may be before, during, or after IL-17RA and IL-17RC form an IL-17RA-IL-17RC heteromeric receptor complex.

Additional embodiments include a method of screening for an antagonist of IL-17RA-IL-17RC heteromeric receptor complex activation, comprising providing an IL-17RA and an IL-17RC in an IL-17RA-IL-17RC heteromeric receptor complex; exposing a candidate agent to said receptor complex; adding one or more IL-17 ligands; and determining the amount of IL-17RA-IL-17RC heteromeric receptor complex activation relative to not having been exposed to the candidate agent. Candidate agents that decrease IL-17RA-IL-17RC heteromeric receptor complex activation in the presence of one or more IL-17 ligands, as measured by a biologically relevant readout (see below), are considered positive. The IL-17 ligand may be IL-17A, IL-17F, or any other IL-17 ligand that binds and activates IL-17RA, IL-17RC, or the IL-17RA-IL-17RC heteromeric receptor complex. Activation is defined elsewhere in the specification. Relevant biological readouts include IL-6, IL-8, CXCL1, CXCL2, GM-CSF, G-CSF, M-CSF, IL-1β, TNFα, RANK-L, LIF, PGE2, IL-12, MMP3, MMP9, GROα, NO, as well as any other molecule known in the art to be released from any cells expressing IL-17RA and/or IL-17RC. The step of exposing a candidate agent to the receptor complex may be before, during, or after IL-17RA and IL-17RC form an IL-17RA-IL-17RC heteromeric receptor complex. It is understood that a candidate agent may partially inhibit IL-17RA-IL-17RC heteromeric receptor complex, i.e., less than 100% inhibition. Under certain assay conditions a candidate agent may completely inhibit IL-17RA-IL-17RC heteromeric receptor complex.

In one aspect, the invention provides for cell-based assays to detect the effect of candidate agents on the association of IL-17RA and IL-17RC, the 17RA-IL-17RC heteromeric receptor complex, as well as activation of the 17RA-IL-17RC heteromeric receptor complex. Thus the invention provides for the addition of candidate agents to cells to screen for 17RA-IL-17RC heteromeric receptor complex antagonists.

By “candidate agent” or “candidate drug” as used herein describes any molecule, such as but not limited to peptides, fusion proteins of peptides (e.g., peptides that bind IL-17RA, IL-17RC, or the 17RA-IL-17RC heteromeric receptor complex that are covalently or non-covalently bound to other proteins, such as fragments of antibodies or protein-based scaffolds known in the art), proteins, antibodies, small organic molecules including known drugs and drug candidates, polysaccharides, fatty acids, vaccines, nucleic acids, etc. that can be screened for activity as outlined herein.

Candidate agents encompass numerous chemical classes. In one embodiment, the candidate agent is an organic molecule, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In alternative embodiments, the candidate bioactive agents may be proteins or fragments of proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

In some embodiments, the candidate agents are peptides. In this embodiment, it can be useful to use peptide constructs that include a presentation structure. By “presentation structure” or grammatical equivalents herein is meant a sequence, which, when fused to candidate bioactive agents, causes the candidate agents to assume a conformationally restricted form. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present randomized peptide structures. Preferred presentation structures maximize accessibility to the peptide by presenting it on an exterior loop. Accordingly, suitable presentation structures include, but are not limited to, minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc. See U.S. Pat. No. 6,153,380, incorporated by reference.

Of particular use in screening assays are phage display libraries; see e.g., U.S. Pat. Nos. 5,223,409; 5,403,484; 5,571,698; and 5,837,500, all of which are expressly incorporated by reference in their entirety for phage display methods and constructs. In general, phage display libraries can utilize synthetic protein (e.g. peptide) inserts, or can utilize genomic, cDNA, etc. digests.

Depending on the assay and desired outcome, a wide variety of cell types may be used, including eukaryotic and prokaryotic cells, with mammalian cells, and human cells, finding particular use in the invention. In one embodiment, the cells may be genetically engineered, for example they may contain exogenous nucleic acids, such as those encoding IL-17RA and IL-17RC. In some instances, the IL-17RA and IL-17RC proteins of the invention are engineered to include labels such as epitope tags, such as but not limited to those for use in immunoprecipitation assays or for other uses.

The candidate agents are added to the cells and allowed to incubate for a suitable period of time. The step of exposing a candidate agent to the receptor complex may be before, during, or after IL-17RA and IL-17RC form an IL-17RA-IL-17RC heteromeric receptor complex. In one embodiment, the association of IL-17RA and IL-17RC is evaluated in the presence and absence of the candidate agents. For example, by using tagged constructs and antibodies, immunoprecipitation experiments can be done. Candidate agents that interfere with IL-17RA and IL-17RC association are then tested for IL-17 ligand family member (such as IL-17A and IL-17F) signaling activity, such as by testing for expression of genes that are activated by IL-17 ligand family member, as mentioned above.

In some embodiments, the IL-17RA and/or IL-17RC proteins are fusion proteins. For example, receptor proteins may be modified in a way to form chimeric molecules comprising an apoprotein fused to another, heterologous polypeptide or amino acid sequence. In one embodiment, such a chimeric molecule comprises a fusion of a receptor with a tag polypeptide which provides an epitope to which an anti-tag antibody can selectively bind. The epitope tag is generally placed at the amino- or carboxyl-terminus of the receptor protein. The presence of such epitope-tagged forms of the receptor can be detected using an antibody against the tag polypeptide. Also, provision of the epitope tag enables the receptor polypeptide to be readily purified by affinity purification using an anti-tag antibody or another type of affinity matrix that binds to the epitope tag. These epitope tags can be used for immobilization to a solid support, as outlined herein.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the FLAGG™-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In some embodiments, the inhibition of binding to IL-17RA-IL-17RC heteromeric receptor complex assays are run in vitro. For example, components of the assay mixture (candidate agent, IL-17RA and IL-17RC) are immobilized on a surface, the other components are added (one of which is labeled in some embodiments). For example, IL-17RA or IL-17RC can be attached to a surface, a candidate agent and a labeled IL-17RA and/or IL-17RC is added. After washing, the presence of the label is evaluated. In this embodiment, the IL-17RA and IL-17RC proteins are isolated as is known in the art. A variety of expression vectors can be made. The expression vectors may be either self-replicating extrachromosomal vectors or vectors which integrate into a host genome. Generally, these expression vectors include transcriptional and translational regulatory nucleic acid operably linked to the nucleic acid encoding the metalloprotein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

In general, attachment will generally be done as is known in the art, and will depend on the composition of the two materials to be attached. In general, attachment linkers are utilized through the use of functional groups on each component that can then be used for attachment. Preferred functional groups for attachment are amino groups, carboxy groups, oxo groups, hydroxyl groups and thiol groups. These functional groups can then be attached, either directly or indirectly through the use of a linker. Linkers are well known in the art; for example, homo- or hetero-bifunctional linkers as are well known (see 1994 Pierce Chemical Company catalog, technical section on cross-linkers, pages 155-200, incorporated herein by reference). Preferred attachment linkers include, but are not limited to, alkyl groups (including substituted alkyl groups and alkyl groups containing heteroatom moieties), with short alkyl groups, esters, amide, amine, epoxy groups and ethylene glycol and derivatives being preferred. Alternatively, fusion partners are used; suitable fusion partners include other immobilization components, such as histidine tags for attachment to surfaces with nickel, functional components for the attachment of linkers and labels, etc., and proteinaceous labels.

In one embodiment, particularly when the candidate agents are immobilized on a solid support, a suitable fusion partner is an autofluorescent protein label. Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

The insoluble supports may be made of any composition to which the compositions can be bound, is readily separated from soluble material, and is otherwise compatible with the overall method of screening. The surface of such supports may be solid or porous and of any convenient shape. Examples of suitable supports include microtiter plates, arrays, membranes and beads, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica based materials including silicon and modified silicon, carbon, metals, inorganic glasses, plastics, ceramics, and a variety of other polymers. In a some embodiments, the solid supports allow optical detection and do not themselves appreciably fluoresce. In addition, as is known the art, the solid support may be coated with any number of materials, including polymers, such as dextrans, acrylamides, gelatins, agarose, etc. Exemplary solid supports include silicon, glass, polystyrene and other plastics and acrylics. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable.

The candidate agents are contacted with the other components of the assay under reaction conditions that favor agent-target interactions. Generally, this will be physiological conditions. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away, in the case of solid phase assays. Assay formats are discussed below.

A variety of other reagents may be included in the assays. These include reagents like salts, neutral proteins, e.g. albumin, detergents, etc which may be used to facilitate optimal apoprotein-agent binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

In one embodiment, any of the assays outlined herein can utilize robotic systems for high throughput screening. Many systems are generally directed to the use of 96 (or more) well microtiter plates, but as will be appreciated by those in the art, any number of different plates or configurations may be used. In addition, any or all of the steps outlined herein may be automated; thus, for example, the systems may be completely or partially automated.

As will be appreciated by those in the art, there are a wide variety of components which may be used, including, but not limited to, one or more robotic arms; plate handlers for the positioning of microplates; automated lid handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In one embodiment, chemically derivatized particles, plates, tubes, magnetic particle, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In one embodiment, platforms for multi-well plates, multi-tubes, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, electroporator, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4° C. to 100° C.

In some preferred embodiments, the instrumentation will include a detector, which may be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluoroescence resonance energy transfer (FRET), SPR systems, luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

The 17RA-IL-17RC heteromeric receptor complex is the biologically active form of the receptor and has been shown herein to respond to ligand-specific activation and release of proinflammatory mediators. It is known in the art that various disease states, as exemplified herein, are associated with increased physiological levels of IL-17 ligand family members. In one embodiment, the IL-17RA-IL-17RC antigen binding proteins are useful for detecting IL-17RA-IL-17RC heteromeric receptor complexes in biological samples and identification of cells or tissues that express said complex. This would be of considerable value to the research community.

The antigen binding proteins of the invention can be used for diagnostic purposes to detect, diagnose, or monitor diseases and/or conditions associated with IL-17 or the IL-17RA or IL-17RC receptor. The invention provides for the detection of the presence of the IL-17 receptor in a sample using classical immunohistological methods known to those of skill in the art (e.g., Tijssen, 1993, Practice and Theory of Enzyme Immunoassays, vol 15 (Eds R. H. Burdon and P. H. van Knippenberg, Elsevier, Amsterdam); Zola, 1987, Monoclonal Antibodies: A Manual of Techniques, pp. 147-158 (CRC Press, Inc.); Jalkanen et al., 1985, J. Cell. Biol. 101:976-985; Jalkanen et al., 1987, J. Cell Biol. 105:3087-3096). The detection of the IL-17 receptor can be performed in vivo or in vitro.

Diagnostic applications provided herein include use of the antigen binding proteins to detect expression of the IL-17 IL-17RA and IL-17RC proteins and binding of the ligands to the IL-17 receptor. Examples of methods useful in the detection of the presence of the IL-17 receptor include immunoassays, such as the enzyme linked immunosorbent assay (ELISA) and the radioimmunoassay (RIA). As outlined above, the use of co-immunoprecipitation is very useful to detect the IL-17RA-IL-17RC heteromeric receptor complex. For diagnostic applications, the antigen binding protein typically may be labeled with a detectable labeling group as defined herein.

One aspect of the invention provides for identifying a cell or cells that express the IL-17RA-IL-17RC heteromeric receptor complex. In a specific embodiment, the antigen binding protein is labeled with a labeling group and the binding of the labeled antigen binding protein to the IL-17 receptor is detected. In a further specific embodiment, the binding of the antigen binding protein to the IL-17 receptor detected in vivo. In a further specific embodiment, the antigen binding protein-IL-17 receptor is isolated and measured using techniques known in the art. See, for example, Harlow and Lane, 1988, Antibodies: A Laboratory Manual, New York: Cold Spring Harbor (ed. 1991 and periodic supplements); John E. Coligan, ed., 1993, Current Protocols In Immunology New York: John Wiley & Sons.

Making Antigen Binding Proteins

Suitable host cells for expression of IL-17RA-IL-17RC antigen binding proteins include prokaryotes, yeast, or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, New York, (1985). Cell-free translation systems could also be employed to produce LDCAM polypeptides using RNAs derived from DNA constructs disclosed herein.

Prokaryotes include gram negative or gram positive organisms, for example, E. coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, an IL-17RA-IL-17RC antigen binding protein may include an N-terminal methionine residue to facilitate expression of the recombinant polypeptide in the prokaryotic host cell. The N-terminal Met may be cleaved from the expressed recombinant IL-17RA-IL-17RC antigen binding protein.

IL-17RA-IL-17RC antigen binding proteins may be expressed in yeast host cells, preferably from the Saccharomyces genus (e.g., S. cerevisiae). Other genera of yeast, such as Pichia, K. lactis or Kluyveromyces, may also be employed. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Hitzeman, EPA-73,657 or in Fleer et. al., Gene, 107:285-195 (1991); and van den Berg et. al., Bio/Technology, 8:135-139 (1990). Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli may be constructed by inserting DNA sequences from pBR322 for selection and replication in E. coli (Amp^{r} gene and origin of replication) into the above-described yeast vectors.

The yeast α-factor leader sequence may be employed to direct secretion of the IL-17RA-IL-17RC antigen binding protein. The α-factor leader sequence is often inserted between the promoter sequence and the structural gene sequence. See, e.g., Kurjan et al., Cell 30:933, 1982; Bitter et al., Proc. Natl. Acad. Sci. USA 81:5330, 1984; U.S. Pat. No. 4,546,082; and EP 324,274. Other leader sequences suitable for facilitating secretion of recombinant polypeptides from yeast hosts are known to those of skill in the art. A leader sequence may be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978. The Hinnen et al. protocol selects for Trp⁺ transformants in a selective medium, wherein the selective medium consists of 0.67% yeast nitrogen base, 0.5% casamino acids, 2% glucose, 10 μg/ml adenine and 20 μg/ml uracil. Yeast host cells transformed by vectors containing ADH2 promoter sequence may be grown for inducing expression in a “rich” medium. An example of a rich medium is one consisting of 1% yeast extract, 2% peptone, and 1% glucose supplemented with 80 μg/ml adenine and 80 μg/ml uracil. Derepression of the ADH2 promoter occurs when glucose is exhausted from the medium.

Mammalian or insect host cell culture systems could also be employed to express recombinant IL-17RA-IL-17RC antigen binding proteins. Baculovirus systems for production of heterologous proteins in insect cells are reviewed by Luckow and Summers, Bio/Technology 6:47 (1988). Established cell lines of mammalian origin also may be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV-1/EBNA-1 cell line derived from the African green monkey kidney cell line CVI (ATCC CCL 70) as described by McMahan et al. (EMBO J. 10: 2821, 1991).

Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide other genetic elements for expression of a structural gene sequence in a mammalian host cell. Viral early and late promoters are particularly useful because both are easily obtained from a viral genome as a fragment which may also contain a viral origin of replication (Fiers et al., Nature 273:113, 1978). Smaller or larger SV40 fragments may also be used, provided the approximately 250 by sequence extending from the Hind III site toward the Bgl I site located in the SV40 viral origin of replication site is included.

Exemplary expression vectors for use in mammalian host cells can be constructed as disclosed by Okayama and Berg (Mol. Cell. Biol. 3:280, 1983). A useful system for stable high level expression of mammalian cDNAs in C127 murine mammary epithelial cells can be constructed substantially as described by Cosman et al. (Mol. Immunol. 23:935, 1986). A useful high expression vector, PMLSV N1/N4, described by Cosman et al., Nature 312:768, 1984 has been deposited as ATCC 39890. Additional useful mammalian expression vectors are described in EP-A-0367566, and in U.S. patent application Ser. No. 07/701,415, filed May 16, 1991, incorporated by reference herein. The vectors may be derived from retroviruses. In place of the native signal sequence, and in addition to an initiator methionine, a heterologous signal sequence may be added, such as the signal sequence for IL-7 described in U.S. Pat. No. 4,965,195; the signal sequence for IL-2 receptor described in Cosman et al., Nature 312:768 (1984); the IL-4 signal peptide described in EP 367,566; the type I IL-1 receptor signal peptide described in U.S. Pat. No. 4,968,607; and the type II IL-1 receptor signal peptide described in EP 460,846.

IL-17RA-IL-17RC antigen binding proteins, as an isolated, purified or homogeneous protein according to the invention, may be produced by recombinant expression systems as described above or purified from naturally occurring cells. IL-17RA-IL-17RC antigen binding proteins can be purified to substantial homogeneity, as indicated by a single protein band upon analysis by SDS-polyacrylamide gel electrophoresis (SDS-PAGE).

One process for producing IL-17RA-IL-17RC antigen binding proteins comprises culturing a host cell transformed with an expression vector comprising a DNA sequence that encodes at least one IL-17RA-IL-17RC antigen binding protein under conditions sufficient to promote expression of said IL-17RA-IL-17RC antigen binding protein. IL-17RA-IL-17RC antigen binding protein is then recovered from culture medium or cell extracts, depending upon the expression system employed. As is known to the skilled artisan, procedures for purifying a recombinant protein will vary according to such factors as the type of host cells employed and whether or not the recombinant protein is secreted into the culture medium. For example, when expression systems that secrete the recombinant protein are employed, the culture medium first may be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Finally, one or more reversed-phase high performance liquid chromatography (RP-HPLC) steps employing hydrophobic RP-HPLC media, (e.g., silica gel having pendant methyl or other aliphatic groups) can be employed to further purify IL-17RA-IL-17RC antigen binding proteins. Some or all of the foregoing purification steps, in various combinations, are well known and can be employed to provide a substantially homogeneous recombinant protein.

It is possible to utilize an affinity column comprising the IL-17RA, or IL-17RC, or both IL-17RA and IL-17RC, or a IL-17RA-IL-17RC heteromeric receptor complex proteins to affinity-purify expressed IL-17RA-IL-17RC antigen binding proteins. IL-17RA-IL-17RC antigen binding proteins can be removed from an affinity column using conventional techniques, e.g., in a high salt elution buffer and then dialyzed into a lower salt buffer for use or by changing pH or other components depending on the affinity matrix utilized. Alternatively, the affinity column may comprise an antibody that binds IL-17RA-IL-17RC antigen binding proteins.

Recombinant protein produced in bacterial culture can be isolated by initial disruption of the host cells, centrifugation, extraction from cell pellets if an insoluble polypeptide, or from the supernatant fluid if a soluble polypeptide, followed by one or more concentration, salting-out, ion exchange, affinity purification or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

Transformed yeast host cells may be employed to express IL-17RA-IL-17RC antigen binding proteins as a secreted polypeptide in order to simplify purification. Secreted recombinant polypeptide from a yeast host cell fermentation can be purified by methods analogous to those disclosed by Urdal et al. 1984, J. Chromatog. 296:171. Urdal et al. describe two sequential, reversed-phase HPLC steps for purification of recombinant human IL-2 on a preparative HPLC column.

### EXAMPLES

Murine and human IL-17A and IL-17F, murine TNF, polyclonals against IL-17RA and IL-17RC and all ELISA kits were obtained from R & D Systems (Minneapolis, Minn.) and used according to manufacturer's specifications. Human IL-17A:Fc and monoclonals against human and mouse IL-17RA were generated as described in (Yao, et al., 1995, Immunity 3: 811-821; Yao, et al., 1995, J. Immunol. 155:5483-5486; Yao, 1997, Cytokine 9:794-800). Multi-analyte profiling of cell culture supernatants was performed by Rules Based Medicine (Austin, Tex.). cDNAs encoding human and mouse IL-17RA have been described previously (see the three Yao references, supra). Human IL-17RC, encoding an open reading frame identical to that previously described (isoform 2, GenBank accession no. NP_{—}703190.1) was obtained from a human pancreas cDNA library using standard techniques known in the art. Human IL-17RA:Fc and human IL-17RC:Fc fusion proteins were prepared essentially as described in Yao (1995 Immunity, supra) using the respective extracellular domains of the receptors.

The generation of C57BL/6 IL-17RA−/− mice has been described previously (Ye, P., et al, 2001 J. Exp. Med. 194:519-527). Primary tail fibrobasts from age and sex matched C57BL/6 and IL-17RA−/− mice were immortalized by transduction with a Simian virus 40 large T-antigen encoding retrovirus as described (Reddy, P., et al., 2000 J. Biol. Chem 275:14608-14614). cDNAs encoding murine and human IL-17RA and IL-17RC were cloned into the mouse stem cell virus (MSCV) retroviral backbone and viruses generated and used according to manafacturer's specifications (Clontech, Mountain View, Calif.). Immortalized fibroblasts were transduced with MSCV viruses encoding IL-17RA or IL-17RC, selected in 5 ug/ml puromycin (InvivoGen, San Diego, Calif.) for 7 days and then sorted for expression by staining with either human IL-17A:Fc or a monoclonal against human IL-17RA (see, Yao 1995 Immunity and Yao 1997 Cytokine, supra). Fibroblasts expressing both IL-17RA and IL-17RC were generated by first transducing IL-17RC and sorting for expression based on human IL-17A:Fc binding followed by transduction of IL-17RA and sorting using monoclonal antiboides against IL-17RA.

The binding affinities of IL-17A and IL-17F to the extracellular domains of human IL-17RA and human IL-17RC fused to the Fc region of human IgG were determined by surface plasmon resonance (BIAcore™) and cell binding analyses. Surface Plasmon Resonance kinetic analysis was performed using a BIAcore 3000™ instrument (Biacore, Inc., Piscataway, N.J.) run at 25° C. To prepare the chip surface, a goat-anti-human IgG Fc gamma-specific capture antibody (Jackson ImmunoResearch, West Grove, Pa.) was immobilized on two flow cells of either a CM4 or CM5 chip following the manufacturer's instructions. 100-300 response units of either human IL-17RA:Fc or human IL-17RC:Fc proteins were captured on one of the two flow cells while the other flow cell was used as a control. Eight concentrations of the analytes (human and murine IL-17A and IL-17F) were injected over the test and control flow cells in the IL-17RA range of 0.041-1500 nM (except for human IL-17F binding to human IL-17RA:Fc which was used at a higher IL-17RA range of 46.9-6000 nM) and allowed to associate for 3-5 minutes and dissociate for 10-30 minutes. Kinetic runs were performed in running buffer (10 mM Hepes, 150 mM NaCl, 3 mM EDTA, 0.005% P-20) with 100 ug/ml BSA added, at a flow rate of IL-17RA of 50 ul/min. The signal from the control flow cell and a buffer only injection cycle were subtracted from sample values to delete “background” for the reference data. After each cycle the flow cells were regenerated using a 30 second pulse of 10 mM glycine, pH 1.5. Data was globally fit to a 1:1 Langmuir model using BiaEvaluation™ software version 4.1 (BIAcore™).

Table 1 shows BIAcore™ data for the binding affinities of immoblized human IL-17RA and IL-17RC to IL-17A and IL-17F.

TABLE 1
Binding affinity^{a} (nM)

| Analyte | huIL-17RA:Fc | huIL-17RC:Fc |
|---|---|---|
| Human IL-17A | 0.5 nM | 0.05 nM |
| Human IL-17F | 100 nM | 0.1 nM |
| Mouse IL-17A | NT^{b} | undetectable |
| Mouse IL-17F | NT^{b} | 0.03 nM |

^{a}k_{off}/kₒₙ as determine by SPR
^{b}not tested

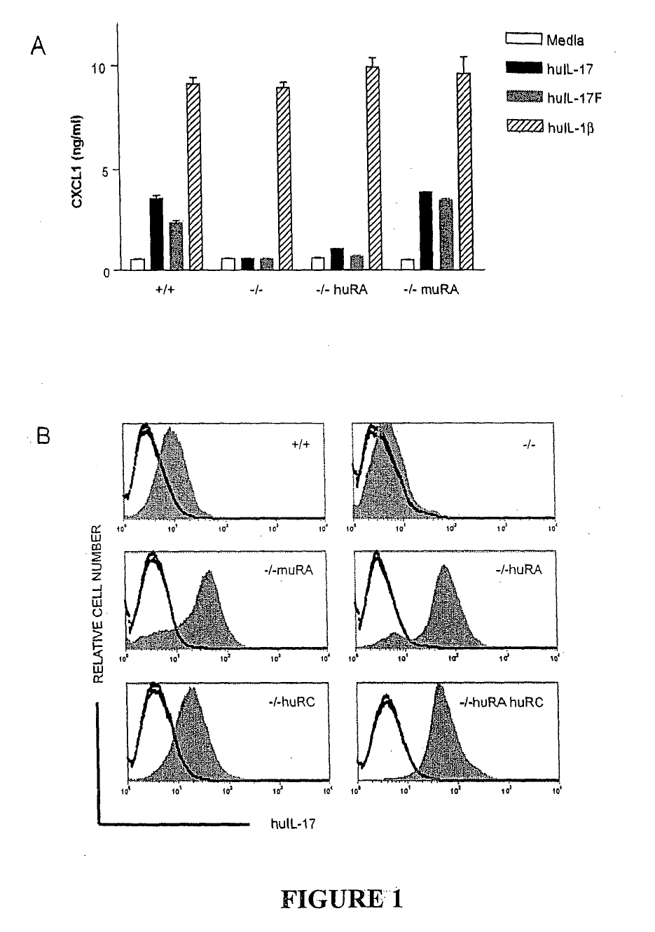

A system was developed where IL-17RA variants could be introduced into IL-17RA−/− fibroblasts and assayed for their ability to induce known IL-17A target genes. IL-17RA+/+ and IL-17RA−/− primary fibroblasts were immortalized by transduction with a Simian virus 40 T-antigen encoding virus. Immortalized IL-17RA+/+ cells, but not IL-17RA−/− cells, are responsive to IL-17A and IL-17F with respect to CXCL1 expression (FIG. 1A). Both populations are responsive to IL-1, indicating that IL-17RA−/− cells are not broadly defective in chemokine expression. Responsiveness was restored in IL-17RA−/− cells stably transduced with a virus encoding mouse IL-17RA. However, IL-17RA−/− cells stably transduced with a virus encoding human IL-17RA responded poorly to human IL-17A and IL-17F (FIG. 1A). Costimulation with TNF, which is known to synergize with IL-17A (Ruddy, et al., 2004, J. Biol. Chem. 279:2559-2567), could not reverse this defect. These defects were not restricted to CXCL1 production, as assessed using a Multi-Analyte panel (Rules Based Medicine, see above) which included the known IL-17A target genes CXCL2, IL-6, and GM-CSF. IL-17RA−/− cells expressing either mouse or human IL-17RA were equally capable of ligand binding (FIG. 1B). These data indicate that human IL-17RA is capable of binding IL-17A when expressed in a mouse cell, but inefficiently delivers IL-17A and IL-17F-induced signals.

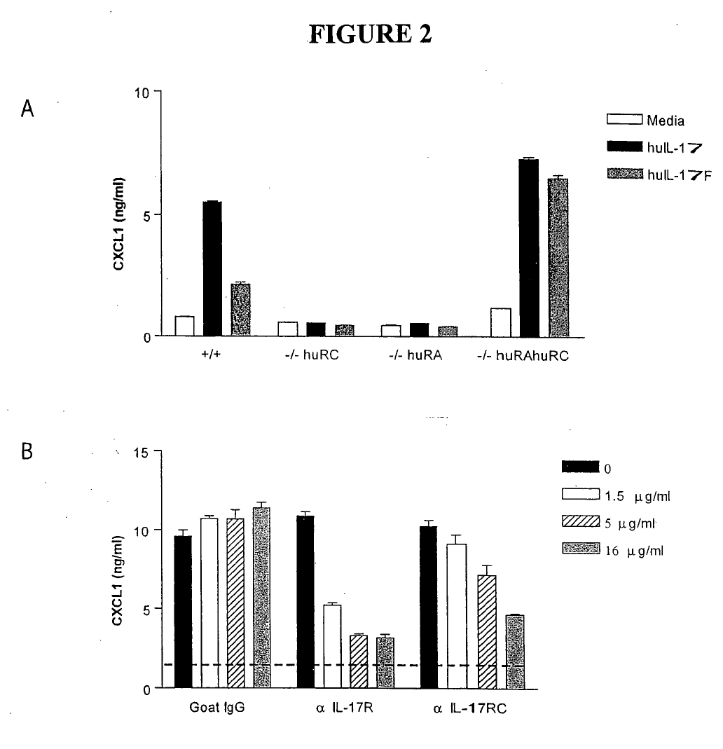

It has been discovered that IL-17RC is an essential component of the IL-17RA complex. Without being bound by theory, one explanation for the inability of human IL-17RA to efficiently signal in a mouse cell is that other members of the IL-17RA family might associate with IL-17RA to mediate IL-17-ligand(s) responsiveness. In such a model, the candidate receptor is expressed on cells known to be responsive to IL-17A. IL-17RC is a widely expressed IL-17R family member (see, Haudenschild, et al., 2002, J. Biol. Chem. 277:4309-4316). BIAcore™ analyses revealed that immobilized human IL-17RC:Fc is capable of binding human IL-17A and IL-17F with high affinity (Table 1). In fact, IL-17RC binds IL-17F with a much higher affinity than does IL-17RA. In agreement with the BIAcore™ data, IL-17RA−/− fibroblasts expressing human IL-17RC are capable of binding IL-17A (FIG. 1B). Surprisingly, the IL-17 ligand binding defect of IL-17RA−/− cells can be “rescued” by transduction with a virus encoding human IL-17RC (FIG. 1B). These results, together with the BIAcore™ analysis, indicate that IL-17A is a potential ligand for IL-17RC. Despite the ability of IL-17RC to bind both IL-17A and IL-17F, IL-17RA−/− fibroblasts expressing human IL-17RC do not produce CXCL1, or any other known IL-17A target genes present on the multi-analyte panel in response to human IL-17A and IL-17F (FIG. 2A and data not shown), suggesting that this receptor is unable to mediate a classical IL-17A signal in the absence of IL-17RA. Strikingly, however, IL-17RA−/− fibroblasts expressing both human IL-17RA and human IL-17RC respond to human IL-17A and IL-17F (FIG. 2A). Thus, the failure of human IL-17RA to signal in a mouse cell can be complemented by co-introduction of human IL-17RC. Mouse fibroblasts express endogenous IL-17RC mRNA, although the observation that IL-17RA−/− cells fail to appreciably bind IL17A suggests that its presence on the cell surface may be low enough to escape detection by flow cytometry using IL-17A:Fc (FIG. 1B).

To further examine a role for IL-17RC in IL-17A and IL-17F signaling in unmanipulated cells, wild type mouse fibroblasts were stimulated with mouse IL-17A in the presence of antibodies against either IL-17RA or IL-17RC. As shown in FIG. 2B, a polyclonal antibody against mouse IL-17RC can inhibit IL-17A responsiveness in a dose dependent manner. This polyclonal antibody does not inhibit IL-1 responsiveness, nor does it block binding of IL-17A to mouse IL-17RA (data not shown), in agreement with the considerable sequence divergence between IL-17RA and IL-17RC. Thus, antibodies against either IL-17RA or IL-17RC can specifically inhibit the activity of IL-17A on mouse fibroblasts.

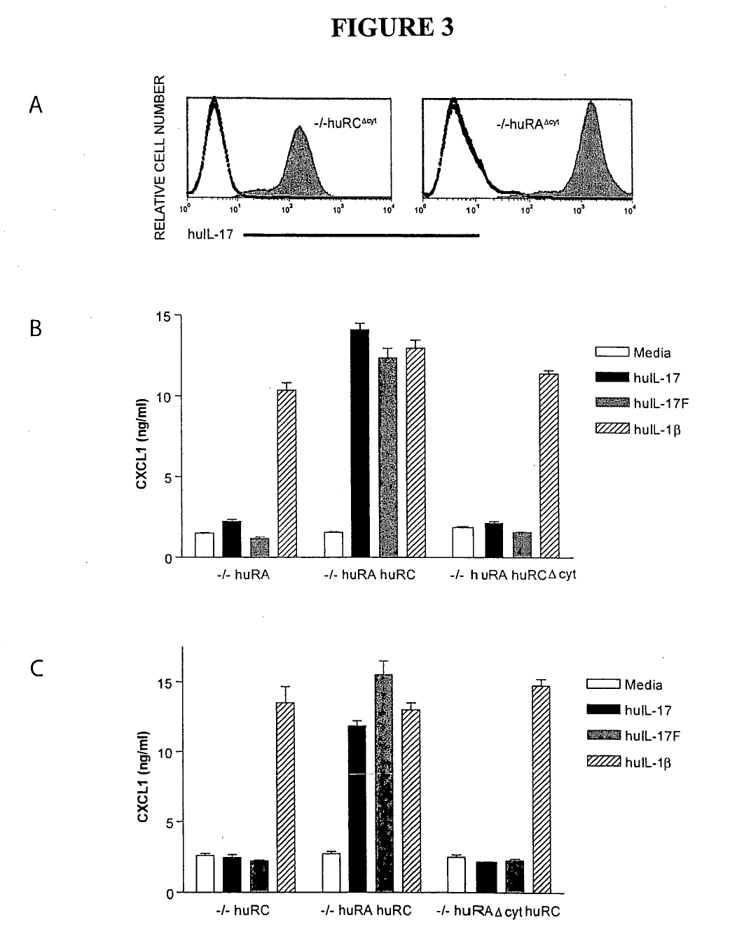

It has also been discovered that the cytoplasmic domains of both IL-17RA and IL-17RC are required for signaling. To assess the roles of the respective receptor cytoplasmic domains in IL-17A signaling, IL-17RA−/− cells were transduced with either full length human IL-17RA plus a cytoplasmic deletion variant of IL-17RC (IL-17RCΔcyt) or with a cytoplasmic deletion variant of human IL-17RA (IL-17RAΔcyt) plus full length IL-17RC. The transduced cytoplasmic deletion variants are both capable of binding IL-17A at the cell surface, as assessed by flow cytometry (FIG. 3A). However, the cytoplasmic domains of both IL-17RA and IL-17RC are essential for mediating IL-17A and IL-17F responsiveness (FIGS. 3B and C).

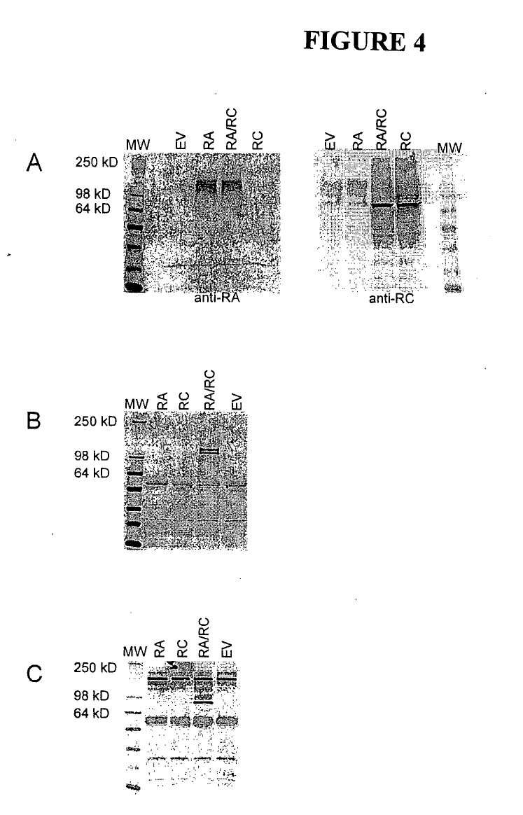

It has been discovered that IL17RA and IL17RC physically associate. IL-17-ligand signaling is mediated by a receptor complex composed of both IL-17RA and IL-17RC subunits. To determine if IL-17RA and IL-17RC physically associate within cells, native human IL-17RA and C-terminally flag-tagged human IL-17RC were overexpressed in HEK 293 cells. Expression of each subunit was confirmed by Western blot analyses (FIG. 4A).

HEK 293 cells were transfected with expression constructs encoding human IL-17RA and/or carboxy-terminal flag tagged human IL-17RC. Cells were lysed in PBS containing 1% (v/v) Triton X-100® (Union Carbide Corp. Danbury Conn., available through Sigma-Aldrich), and Complete Proteinase Cocktail™ (Roche, Mannheim, Germany). Lysates were immunoprecipitated on ice for 2 hours with either 20 ug anti-flag M2 monoclonal or 20 ug anti-human IL-17RA monoclonal (see above for reference). Immunoprecipitates were collected on protein A+G Ultralink™ beads (Pierce, Rockford, Ill.). Beads were washed in lysis buffer and the material resolved on 4-20% SDS-PAGE and Western blotted with goat polyclonals against either human IL-17RC or human IL-17RA (R & D Systems, Minneapolis, Minn.) and developed with an Alexa Fluor 680™ conjugated anti-goat antibody (Invitrogen Life Technologies, San Diego, Calif.).

Human IL-17RA is present in immunoprecipitates brought down with the anti-flag monoclonal M2 used to immunoprecipitate IL-17RC (FIG. 4B) and IL-17RC is present in immunoprecipitates brought down with an antibody against IL-17RA (FIG. 4C). Thus, IL-17RA and IL-17RC are capable of associating in vitro, supporting a model in which IL-17A and IL-17F signaling is mediated by a heteromeric receptor complex containing, minimally, IL-17RA and IL-17RC chains.

Signaling triggered by ligand-induced activation of type 1 receptors is dependent upon receptor subunit association (Lemmon, et al., 1994, Trends Biochem. Sci. 19:459-463). In the case of receptor homodimerization, ligand independent signaling can be achieved by forced receptor overexpression. Ligand-independent chemokine expression is not observed in fibroblasts overexpressing either IL-17RA or IL-17RC alone (FIG. 2A). The observation that forced co-expression of IL-17RA and IL-17RC is also insufficient to drive ligand-independent signaling (FIG. 2A) suggests the possibility that yet additional receptor components are involved. Alternatively, ligand-induced receptor conformational changes may be essential for activity irrespective of receptor density. IL-17RA was recently shown to self-associate on the cell surface in the absence of ligand (Kramer, et al., 2006, J. Immunol 176:711-715). Interestingly, association was reduced in the presence of IL-17A. One intriguing explanation for these data is that IL-17RA is maintained in an inactive, homotypic state in the absence of IL-17A. It is plausible that ligand binding alters the conformation of IL-17RA to favor a productive, heterotypic interaction with IL-17RC.

IL-17RC was recently shown to protect cells from TNF induced apoptosis when overexpressed in transfected human cells (You, et al., 2006, Cancer Res. 66:175-183). This protection is not mediated through canonical survival pathways and is apparently not dependent upon known IL-17 family ligands. Additionally, the panel of genes reported to be activated in IL-17RC overexpressing cells does not include any of the inflammatory chemokines and cytokines classically associated with IL-17R activation (Ibid). These results suggest that IL-17RC may have biologic functions independent of IL-17RA. Similarly, cross-linking of over-expressed IL-17RE, a newly described orphan member of the IL-17R family, can activate a mitogenic pathway (Li, et al., 2005, Cell. Signal. 18:1287-1298). Thus, the cellular outcomes of signaling through IL-17R family receptors are likely dependent upon both homotypic and heterotypic receptor interactions. The results herein demonstrate that such heterotypic interactions involve multiple IL-17R family members (e.g., IL-17RA and IL-17RC), and the various embodiments of the invention provide a potential framework for elucidating the ligand specificities and biologic activities of current orphan IL-17 and IL-17R family members. Equally important, the data presented herein shows that inhibition of the formation of the IL-17RA-IL-17RC heteromeric receptor complex can inhibit receptor activation, which in turn would reduce the proinflammatory responses of IL-17RA and/or IL-17RC activation.

Monoclonal Antibodies:

This example illustrates a method for preparing monoclonal antibodies to a IL-17RA-IL-17RC heteromeric receptor complex. Purified IL-17RA-IL-17RC heteromeric receptor complex, a fragment thereof such as the coupled extracellular domains, synthetic peptides or cells that express important association domains of the IL-17RA-IL-17RC heteromeric receptor complex can be used to generate monoclonal antibodies against the IL-17RA-IL-17RC heteromeric receptor complex using conventional techniques, for example, those techniques described in U.S. Pat. No. 4,411,993. Briefly, mice are immunized with IL-17RA-IL-17RC heteromeric receptor complex as an immunogen emulsified in complete Freund's adjuvant, or any other suitable adjuvant, and injected in amounts ranging from 10-100 μg subcutaneously or intraperitoneally. Ten to twelve days later, the immunized animals are boosted with additional IL-17RA-IL-17RC heteromeric receptor complex emulsified in incomplete Freund's adjuvant, or any other suitable adjuvant. Mice are periodically boosted thereafter on a weekly to bi-weekly immunization schedule. Serum samples are periodically taken by retro-orbital bleeding or tail-tip excision to test for IL-17RA-IL-17RC heteromeric receptor complex antibodies by dot blot assay or ELISA (Enzyme-Linked Immunosorbent Assay).

Following detection of an appropriate antibody titer, positive animals are provided one last intravenous injection of IL-17RA-IL-17RC heteromeric receptor complex in saline, for example. Three to four days later, the animals are sacrificed, spleen cells harvested, and spleen cells are fused to a murine myeloma cell line, such as but not limited to NS1 or preferably P3x63Ag8.653 (ATCC CRL 1580). Fusions generate hybridoma cells, which are plated in multiple microtiter plates in a HAT (hypoxanthine, aminopterin and thymidine) selective medium to inhibit proliferation of non-fused cells, myeloma hybrids, and spleen cell hybrids.

The hybridoma cells are screened by ELISA for reactivity against purified methods known in the art. Positive hybridoma cells can be injected intraperitoneally into syngeneic mice to produce ascites containing high concentrations of anti-IL-17RA-IL-17RC heteromeric receptor complex monoclonal antibodies. Alternatively, hybridoma cells can be grown in vitro in flasks or roller bottles by various techniques. Monoclonal antibodies produced in mouse ascites can be purified by any method known in the art, such as ammonium sulfate precipitation, followed by gel exclusion chromatography.

1-77. (canceled)

78. An isolated human monoclonal antibody or antigen-binding fragment thereof that inhibits IL-17RA and IL-17RC from forming an IL-17RA-IL-17RC heteromeric receptor complex.

79. The antibody or fragment thereof of claim 78, wherein said antibody or antigen-binding fragment thereof binds IL-17RA and inhibits the association of IL-17RA and IL-17RC.

80. The antibody or fragment thereof of claim 78, wherein said antibody or antigen-binding fragment thereof binds IL-17RC and inhibits the association of IL-17RA and IL-17RC.

81. The antibody or antigen-binding fragment thereof of claim 78, wherein said antibody or fragment thereof binds both IL-17RA and IL-17RC and inhibits the association of IL-17RA and IL-17RC in forming a IL-17RA-IL-17RC heteromeric receptor complex.

82. An isolated human monoclonal antibody or antigen-binding fragment thereof that inhibits activation of the IL-17RA-IL-17RC heteromeric receptor complex.

83. The antibody or antigen-binding fragment thereof of claim 82, wherein said antibody or fragment thereof binds IL-17RA and inhibits activation of the IL-17RA-IL-17RC heteromeric receptor complex.

84. The antibody or antigen-binding fragment thereof of claim 82, wherein said antibody or fragment thereof binds IL-17RC and inhibits activation of the IL-17RA-IL-17RC heteromeric receptor complex.

85. The antibody or antigen-binding fragment thereof of claim 82, wherein said antibody or fragment thereof binds both IL-17RA and IL-17RC and inhibits activation of the IL-17RA-IL-17RC heteromeric receptor complex.

86. The antibody or antigen-binding fragment thereof of claim 82, wherein said antibody prevents IL-17A and/or IL-17F from activating the IL-17RA-IL-17RC heteromeric receptor complex.

87. A method of inhibiting IL-17RA-IL-17RC heteromeric receptor complex formation in vivo, comprising exposing a cell expressing IL-17RA, IL-17RC, and IL-17RA-IL-17RC heteromeric receptor complex to an IL-17RA-IL-17RC antagonistic antibody or antigen-binding fragment thereof, such that IL-17RA and IL-17RC are partially or fully inhibited from forming an IL-17RA-IL-17RC heteromeric receptor complex.

88. A method of inhibiting IL-17RA-IL-17RC heteromeric receptor complex activation in vivo, comprising exposing a cell expressing IL-17RA, IL-17RC, and IL-17RA-IL-17RC heteromeric receptor complex to an IL-17RA-IL-17RC antagonistic antibody, or antigen-binding fragment thereof, such that IL-17A and/or IL-17F are partially or fully inhibited from activating said IL-17RA-IL-17RC heteromeric receptor complex.

89. A method of reducing at least one proinflammatory mediator in a patient having an autoimmune disease, an inflammatory disease, rheumatoid arthritis, inflammatory bowel disease, psoriasis, asthma, or chronic obstructive pulmonary disease, comprising administering an IL-17RA-IL-17RC antagonistic antibody, or antigen-binding fragment thereof, to said patient, wherein said patient's cells express IL-17RA, IL-17RC, and IL-17RA-IL-17RC heteromeric receptor complex, and wherein said antagonistic antibody, or antigen-binding fragment thereof, partially or fully inhibits IL-17A and/or IL-17F from activating said IL-17RA-IL-17RC heteromeric receptor complex, wherein said proinflammatory mediator is selected from the group consisting of: IL-6, IL-8, CXCL1, CXCL2, GM-CSF, G-CSF, M-CSF, IL-1β, TNFα, RANK-L, LIF, PGE2, IL-12, MMP3, MMP9, GROα, and NO.

90. A method of treating an autoimmune disease, an inflammatory disease, rheumatoid arthritis, inflammatory bowel disease, psoriasis, asthma, or chronic obstructive pulmonary disease, comprising administering an IL-17RA-IL-17RC antagonistic antibody, or antigen-binding fragment thereof, to a patient in need thereof, wherein said patient's cells express IL-17RA, IL-17RC, and IL-17RA-IL-17RC heteromeric receptor complex, and wherein said antagonistic antibody, or antigen-binding fragment thereof, partially or fully inhibits IL-17A and/or IL-17F from activating said IL-17RA-IL-17RC heteromeric receptor complex.
